(19) United States
(12) Patent Application Publication
(10) Pub. No.: US 2023/0270444 A1
(43) Pub. Date: Aug. 31, 2023

(54) CALIBRATION OF SOLID-STATE SENSORS

(71) Applicants: Certus Critical Care, Inc., Salt Lake City, UT (US); Wake Forest University Health Sciences, Winston-Salem, NC (US)

(72) Inventors: Daniel FONG, Sacramento, CA (US); Timothy WILLIAMS, Winston-Salem, NC (US); Patrick Ryan KOLBAY, Salt Lake City, UT (US); David POISNER, Carmichael, CA (US); Michael Austin JOHNSON, Holladay, UT (US); Lucas NEFF, Winston-Salem, NC (US); Kobi IKI, San Carlos, CA (US)

(21) Appl. No.: 18/157,759

(22) Filed: Jan. 20, 2023

Related U.S. Application Data
(60) Provisional application No. 63/301,475, filed on Jan. 20, 2022.

(51) Int. Cl. A61B 17/12; A61M 25/10

(52) U.S. Cl. CPC A61B 17/12136; A61M 25/10182; A61B 5/0215

(57) ABSTRACT

A method for calibrating at least one solid-state sensor coupled to an elongate body comprising an expandable member is described herein. In some variations, the method may include advancing the expandable member to a target location in a blood vessel of a patient, injecting a calibration bolus into the expandable member, obtaining, using a controller, a first sensor data from an expandable member sensor, obtaining, using the controller, a second sensor data from the at least one solid-state sensor, and adjusting the second sensor data based on the first sensor data.

### CROSS-REFERENCE TO RELATED APPLICATIONS

This application claims priority to U.S. Provisional Application No. 63/301,475, filed on Jan. 20, 2022, which is hereby incorporated by reference in its entirety.

### GOVERNMENT SUPPORT

This invention was made with government support under grant FA8650-20-2-6116 and W81XWH-21-C-0058 awarded by the United States Air Force/Air Force Material Command. The government has certain rights in the invention.

### TECHNICAL FIELD

This invention relates generally to the field of calibrating one or more solid-state sensors. In particular, this invention relates to the field of calibrating solid-state sensors that may be integrated into a catheter for monitoring physiologic conditions during a medical procedure.

### BACKGROUND

Measuring physiologic conditions, especially in real-time, at a point-of-intervention (e.g., during medical procedure) may provide valuable data that may guide treatment decisions. For example, changes to physiologic conditions during a medical procedure may provide information about the patient, such as for example, whether the patient needs medication, the amount of medication that may be needed, whether the patient needs intravenous (IV) fluids, whether the medical procedure has adversely impacted the patient's blood flow and/or blood pressure, whether the procedure or the medical device needs to be modified and or changed, etc.

To measure physiologic conditions during a medical procedure, catheters are used in conjunction with sensor devices. Some existing catheters include pressure sensors that utilize fluid columns to measure blood pressure. Typically, these pressure sensors are coupled to the catheters but positioned outside the body of the patient or positioned outside the body of the patient as a stand-alone pressure sensor/device that is operatively connected to the catheters. The pressure in the body is transduced through the fluid column within the catheter to the pressure sensor. In these cases, catheters may need to be modified to accommodate the pressure sensing columns. Moreover, fluid columns can be cumbersome to use and may result in inaccurate sensing data. For example, fluid columns are known to clot with blood. Additionally, as the length of the fluid column increases, the sensor signals may begin to dampen, which may result in inaccuracies and an inability to measure the true maximum and minimum values of a cyclical or changing physiologic condition. Additionally, movements of the fluid column and/or inadvertent contact with the fluid column may lead to excessive noise that can result in inaccurate pressure readings. Thus, use of fluid columns can be particularly challenging, especially in circumstances where portability and/or field use are desirable.

In addition to the above, there is significant set up time associated with existing catheters that utilize fluid columns. For instance, during set up, a user (e.g., surgeon, operator, etc.) may have to flush the fluid columns with saline to ensure that there is no air in the fluid column. The user may also have to continuously and/or intermittently continue to flush the fluid columns to ensure that clots are not building up in the fluid columns. This can make such catheters cumbersome to use. The use of fluid columns may be particularly challenging in circumstances where blood pressure measurement and/or pressure monitoring may be needed quickly without significant set up time.

More recently, integration of solid-state sensors into catheters to measure physiologic conditions in real-time have been considered. However, there are several challenges associated with using solid-state sensors to measure physiologic conditions. Solid-state sensors are extremely sensitive to changes in temperature and media (e.g., air, fluid, etc.). Therefore, when catheters with solid-state sensors are transitioned from media to media (air to fluid) or from temperature to temperature, such as inserted into a body of a patient after being outside the body, the transition from air (e.g., outside the body of the patient) to fluid (e.g., fluids inside the body of the patient) may cause rapid changes in sensor readings, thereby leading to erroneous sensor data. Furthermore, the amount of change in the sensor readings obtained from one sensor due to the transition may be different from the amount of change in sensor readings obtained from another similar sensor. Thus, it may be difficult to uniformly calibrate solid-state sensors.

Accordingly, there is a need for sophisticated systems, devices, and methods for calibrating solid-state sensors.

### SUMMARY

Described herein are systems, devices, and methods for calibrating solid-state sensors. The methods may include advancing an elongate body and an expandable member to a target location, injecting a calibration bolus into the expandable member, obtaining data (e.g., waveforms) from the expandable member sensor and one or more solid-state sensors, and adjusting the sensor data from at least one solid-state sensor based on data from the expandable member sensor. Negative pressure (i.e., vacuum) may be applied to the expandable member. The negative pressure may be applied before or during the process of calibrating the solid-state sensors, and may be used as a safeguard (e.g., safety feature) during calibration.

In some variations, a method for calibrating at least one solid-state sensor coupled to an elongate body comprising an expandable member may include advancing the expandable member to a target location in a blood vessel of a patient, injecting a fluid calibration bolus into the expandable member, obtaining, using a controller, a first sensor data from an expandable member sensor, obtaining, using the controller, a second sensor data from the at least one solid-state sensor, and adjusting the second sensor data based on the first sensor data. The first sensor data may represent pressure in the expandable member.

In some variations, adjusting the second sensor data may comprise shifting at least one of a calibration curve and a calibration constant of the at least one solid-state sensor. In some variations, adjusting the second sensor data may further comprise determining a first mean of the first sensor data, determining a second mean of the second sensor data, and adjusting the second mean based on the first mean. In some variations, the first sensor data may include a first waveform and the second sensor data may include a second waveform. Adjusting the second sensor data may comprise adjusting the second waveform based on the first waveform.

In some variations, the second sensor data and the first sensor data may comprise data from the at least one solid-state sensor and data from the expandable member sensor respectively at a point in time. In some variations, the pressure in the expandable member may be indicative of pressure at the target location in the blood vessel. In some variations, the method may further comprise prior to advancing the expandable member, determining a volume of the fluid calibration bolus.

In some variations, the fluid calibration bolus may be based at least in part on one or more of a length of the elongate body. In some variations, injecting the fluid calibration bolus may further comprise injecting the fluid calibration bolus into the expandable member using a syringe pump. A volume of the fluid calibration bolus may be based at least in part on a distance between the syringe pump and the expandable member. In some variations, injecting the fluid calibration bolus may comprise injecting, via a lumen of the elongate body, the fluid calibration bolus into the expandable member. A volume of the fluid calibration bolus may be based at least in part on a volume of the lumen.

In some variations, the volume of the fluid calibration bolus may be between about 1 ml and about 1.5 ml. In some variations, the volume of the fluid calibration bolus may be less than 5% of a volume of the expandable member. In some variations, the volume of the fluid calibration bolus may not distend the expandable member.

In some variations, obtaining the first sensor data and obtaining the second sensor data may include obtaining the first sensor data and the second sensor data after a predetermined amount of time has elapsed from advancing the expandable member. In some variations, the predetermined amount of time may be between about 15 seconds and about 45 seconds.

In some variations, the method may further comprise determining, using the controller, that the expandable member is positioned within the blood vessel. Determining that the expandable member is positioned within the blood vessel may include determining, using the controller, that the expandable member is positioned within the blood vessel based on the second sensor data. The second sensor data may include a pressure waveform obtained from the at least one solid-state sensor. The pressure waveform may represent arterial pressure or venous pressure.

In some variations, determining that the expandable member is positioned within the blood vessel may include receiving, at the controller, user input indicating that the expandable member is positioned within the blood vessel. In some variations, the method may further comprise prior to advancing the expandable member, determining an initial setpoint for the expandable member sensor. In some variations, determining the initial setpoint may further comprise receiving, at the controller, a barometric pressure from a barometric pressure sensor and determining, using the controller, the initial setpoint based at least in part on the barometric pressure. In some variations, determining the initial set-point may include determining a baseline value for the expandable member sensor.

In some variations, the second sensor data from the at least one solid-state sensor may include a first waveform. The method may further comprise determining, based at least in part on the first waveform, whether a volume of the fluid calibration bolus is a non-disruptive volume of fluid. In some variations, the method may further comprise receiving a user input from a user indicating that the fluid calibration bolus has been injected into the expandable member.

In some variations, the second sensor data from the at least one solid-state sensor may include a first waveform. The method may further comprise determining, based at least in part on the first waveform, that a volume of the fluid calibration bolus injected into the expandable member exceeds a non-disruptive volume of fluid. In some variations, the at least one solid-state sensor may include a first pressure sensor proximal to the expandable member and a second pressure sensor distal to the expandable member.

In some variations, adjusting the second sensor data may include adjusting sensor data from the second pressure sensor based on the first sensor data, and adjusting sensor data from the first pressure sensor based on the first sensor data. In some variations, the second sensor data may include third sensor data from the first pressure sensor and fourth sensor data from the second pressure sensor. Adjusting the second sensor data may include adjusting fourth sensor data from the second pressure sensor based on the first sensor data, and adjusting third sensor data from the first pressure sensor based on the fourth sensor data.

A system for measuring a physiological condition in a patient may comprise an elongate body comprising an expandable member and a solid-state sensor, a syringe pump in fluid communication with the expandable member, and a controller comprising an expandable member sensor in fluid communication with the expandable member. The controller may be communicatively coupled to the at least one solid-state sensor and configured to: obtain, from the expandable member sensor, a first sensor data representing pressure at a target location in the patient, obtain, from the solid-state sensor, a second sensor data, and adjust the second sensor data based at least in part on the first sensor data.

In some variations, the controller may further comprise a housing comprising a recessed portion configured to receive the syringe pump. In some variations, the controller may be further configured to actuate the syringe pump to inject a calibration bolus into the expandable member. In other variations, a separate syringe pump may be used to inject the calibration bolus into the expandable member. Put another way, a first syringe pump may be used to meter fluid into and out of the expandable member to control blood flow, and a second, different syringe pump may be used to inject the calibration bolus into the expandable member. The second syringe pump may be operated manually (e.g., actuated by a user by hand, without use of the controller), or by using the controller. In some variations, the controller may be further configured to adjust the second sensor data by shifting a calibration curve of the solid-state sensor.

In some variations, the controller may be further configured to determine a first mean of the first sensor data, determine a second mean of the second sensor data, and adjust the second mean based on the first mean. The first sensor data may include a first waveform and the second sensor data may include a second waveform. The controller may be configured to adjust the second waveform based on the first waveform.

In some variations, the second sensor data and the first sensor data may comprise data from the solid-state sensor and data from the expandable member sensor respectively at a point in time. In some variations, the controller may be further configured to determine whether a calibration bolus has been injected into the expandable member based at least in part on at least one of the second sensor data and the first sensor data. In some variations, a volume of the calibration bolus may be based at least in part on a length of the elongate body. In some variations, a volume of the calibration bolus may be based at least in part on a distance between the syringe pump and the expandable member. In some variations, the elongate body may include a lumen connecting the syringe pump to the expandable member. A volume of the calibration bolus may be based at least in part on a volume of the lumen. In some variations, a volume of the calibration bolus may be between about 1 ml and about 1.5 ml. In some variations, a volume of the calibration bolus may be less than 5% percentage of a volume of the expandable member. In some variations, the calibration bolus may not distend the expandable member.

In some variations, the controller may be configured to determine that the expandable member is positioned within a blood vessel based on the second sensor data. The second sensor data may include a pressure waveform obtained from the solid-state sensor. The pressure waveform may represent arterial pressure or venous pressure. In some variations, the controller may comprise at least one user control to indicate that the expandable member is positioned in a blood vessel of the patient. In some variations, the controller may be configured to determine an initial setpoint for the expandable member.

In some variations, the system may further include a barometric pressure sensor. The controller may be further configured to receive a barometric pressure from the barometric pressure sensor, and determine the initial setpoint based at least in part on the barometric pressure. In some variations, the elongate body may be configured to be advanced into an aorta. In some variations, the controller may be configured to obtain the first sensor data after a calibration bolus has been injected into the expandable member.

A method for calibrating a first solid-state sensor and a second solid-state sensor coupled to an elongate body comprising an expandable member may include advancing the expandable member to a target location in a blood vessel of a patient, obtaining, using a controller, a first sensor data from the first solid-state sensor, obtaining, using the controller, a second sensor data from the second solid-state sensor, determining, using the controller, an inherent offset between the first solid-state sensor and the second solid-state sensor, injecting a calibration bolus into the expandable member, obtaining, using the controller, a third sensor data from an expandable member sensor, adjusting the first sensor data based on the third sensor data, and after adjusting the first sensor data, adjusting the second sensor data based on the first sensor data and the inherent offset. The first solid-state sensor may be positioned distal to the expandable member. The second solid-state sensor may be positioned proximal to the expandable member. The third sensor data may represent pressure at the target location.

A method for calibrating a first solid-state sensor and a second solid-state sensor coupled to an elongate body comprising an expandable member may comprise advancing the expandable member to a target location in a blood vessel of a patient, obtaining, using a controller, a first sensor data from the first solid-state sensor, obtaining, using the controller, a second sensor data from the second solid-state sensor, determining, using the controller, an inherent offset between the first solid-state sensor and the second solid-state sensor, injecting a calibration bolus into the expandable member, obtaining, using the controller, a third sensor data from an expandable member sensor, determining, using the controller, a difference between the first sensor data and the third sensor data to determine a proximal offset, determining, using the controller, a difference between the proximal offset and the inherent offset to determine a distal offset, adjusting a first calibration curve for the first solid-state sensor based on the proximal offset, and adjusting a second calibration curve for the second solid-state sensor based on the distal offset. The first solid-state sensor may be positioned distal to the expandable member. The second solid-state sensor may be positioned proximal to the expandable member. The third sensor data may represent pressure at the target location.

Other methods may include calibrating a system comprising a first solid-state sensor and a second solid-state sensor coupled to an elongate body having an expandable member, by advancing the expandable member to a target location in a blood vessel of a patient; applying, using a controller, a first calibration safeguard to the system; obtaining, using the controller, a first sensor data from the first solid-state sensor, where the first solid-state sensor is positioned proximal to the expandable member; obtaining, using the controller, a second sensor data from the second solid-state sensor, where the second solid-state sensor is positioned distal to the expandable member; determining, using the controller, an inherent offset between the first solid-state sensor and the second solid-state sensor; and injecting a calibration bolus into the expandable member. After injecting the calibration bolus, the method may include applying a second calibration safeguard to the system using the controller; obtaining, using the controller, a third sensor data from an expandable member sensor, the third sensor data representing pressure at the target location; obtaining, using the controller, a tip sensor offset using the third sensor data and data from the first solid-state sensor; and adjusting the first sensor data and the second sensor data based on the tip sensor offset and the inherent offset. Applying the first calibration safeguard may include applying negative pressure to the expandable member, and applying the second calibration safeguard may include measuring the pressure in the expandable member and determining that the pressure in the expandable member is at or above a threshold value. The threshold value may be zero. After calibration, the calibrated sensors of the system may be used to control blood flow in a patient. For example, the blood flow may be controlled to treat one or more types of shock, such as neurogenic shock, hemorrhagic shock, hypovolemic shock, and septic shock.

### BRIEF DESCRIPTION OF THE DRAWINGS

FIG. 1 depicts example sensor readings obtained from the same solid-state sensor before and after sudden exposure to water on three different occasions.

FIG. 2A is a block diagram illustrating an exemplary variation of a system for measuring physiologic conditions.

FIG. 2B illustrates an exemplary variation of a system for measuring physiologic conditions.

FIG. 2C illustrates another exemplary variation of a system for measuring physiologic conditions.

FIG. 3 is a flowchart illustrating an exemplary variation of a method for calibrating solid-state sensors.

FIG. 4 is a plot containing sensor data from an expandable member sensor, a proximal solid-state sensor, and a distal solid-state sensor before calibration, during calibration, and after calibration from an exemplary variation of a system for calibration solid-state sensors.

FIG. 5 illustrates another exemplary variation of sensor data from an expandable member sensor, a proximal solid-state sensor, and a distal solid-state sensor before calibration, during calibration, and after calibration using the systems and methods described herein.

FIG. 6 is a flowchart illustrating an exemplary variation of another method for calibrating solid-state sensors.

FIG. 7 provides data from an expandable member sensor, a proximal solid-state sensor, and a distal solid-state sensor before calibration, during calibration, and after calibration during an animal experiment.

### DETAILED DESCRIPTION

Non-limiting examples of various aspects and variations of the invention are described herein and illustrated in the accompanying drawings.

Systems, devices, and methods for calibrating solid-state sensors are described herein. More specifically, systems, devices, and methods for calibrating one or more sensors coupled to (e.g., attached to, integrated with, mounted on, and/or the like) medical devices comprising an elongate body (e.g., a catheter) for monitoring physiologic conditions are described herein.

Devices comprising an elongate body may be used in various medical procedures, such as for example, cardiovascular procedures, urological procedures, gastrointestinal procedures, neurovascular procedures, ophthalmic procedures, etc. Many of these medical procedures may be assisted by one or more sensors that provide information to a user (e.g., surgeon, operator, etc.) at the point of intervention.

Some existing sensors for providing information at the point of intervention utilize fluid columns to measure pressure. However, fluid columns suffer from several drawbacks such as, for example, excessive noise, over and/or under dampening of the signal, high susceptibility to noise during any movement of the patient or the arterial line, clots and/or blockages in the arterial line, lack of portability, etc.

To overcome the limitations of fluid columns, it may be advantageous to utilize medical devices that are coupled to, or otherwise incorporate, solid-state sensors to measure physiologic conditions. While solid-state sensors are known to provide accurate sensor readings despite being subject to force, movement, and vibrations, they also have certain limitations. One such limitation is the high sensitivity to environmental factors. For instance, solid-state sensors may be susceptible to deformation owing to sudden changes in temperature. Such deformation may result in an erroneous change (e.g., rapid fall or a rapid rise) in the sensor data. This erroneous change may lead to erroneous sensor readings, thereby impacting the accuracy of sensor data obtained from the solid-state sensor. Additionally, solid-state sensors may absorb moisture as they transition from a dry media (e.g., air) to a wet media (e.g., fluid). Such transition may also result in rapid changes to the sensor data, which may result in inaccurate sensor readings.

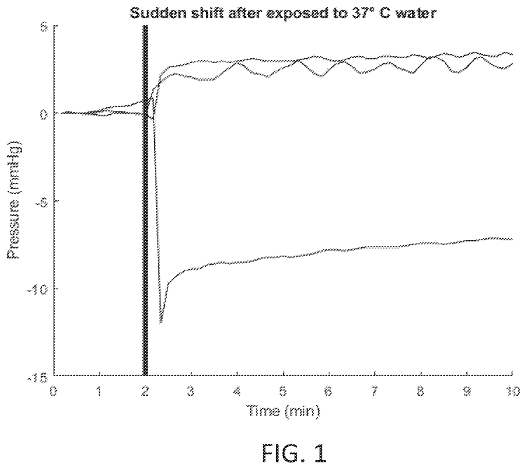

Rapid changes to sensor data may be addressed by accounting for these rapid changes (e.g., rapid change in temperature, sudden transition from one media to another, etc.). For example, determining one or more offsets that may account for these rapid changes and calibrating the solid-state sensors prior to use based on these offsets may improve the accuracy of the sensors. But, characterizing all potential offsets for all possible changes prior to use may be challenging. Furthermore, the magnitude of these offsets may be difficult to predict since these offsets may be sensor specific and may depend on a variety of factors such as ambient temperature, humidity, position of the sensors, etc. For instance, the same solid-state sensor may show different changes when transitioned from air to fluid at different times. FIG. 1 depicts example sensor readings obtained from the same solid-state sensor before and after a sudden transition from one media to another, in this case, water, at three different points in time. More specifically, FIG. 1 depicts pressures measured from a solid-state sensor over a 10 minute period on three different occasions as the solid-state sensor is suddenly exposed to water at 37 degrees Celsius, which occurs at minute two. After the solid-state sensor is exposed to the water, the measured pressures change suddenly, rising in two instances, and falling in one instance. These sudden changes resolve in about thirty seconds after minute two (e.g., thirty seconds after the sudden exposure). However, as seen in FIG. 1, the amount of change in the pressure readings is different for each of the three different occasions. Accordingly, predicting a universal offset for all solid-state sensors to account for the changes in data based on environmental changes may be difficult, which in turn makes it challenging to universally calibrate solid-state sensors to account for these changes. Similarly, two different solid-state sensors of the same type may show different changes in the same environment, further complicating universal calibration. Additionally, if the solid-state sensors were calibrated at the time of manufacturing, subjecting the medical devices to processes such as sterilization before clinically using the medical device may change the calibration offset determined at the time of manufacturing. That is, the offset determined at the time of manufacturing may change from manufacturing to post sterilization when the device is used clinically.

Accordingly, when used in medical devices, it may be beneficial to calibrate solid-state sensors after the medical device has been inserted into a patient's body (i.e., after the solid-state sensors have been exposed to the sudden environmental change). Described herein are systems, devices, and methods for calibrating solid-state sensors after the solid-state sensors have been exposed to a sudden change in an environment. More specifically, the devices, systems, and methods described herein may use sensor data from a first sensor type (e.g., a fluid-column based pressure sensor) to calibrate sensors of a second, different sensor type (e.g., solid-state pressure sensors). The sensor of the first sensor type may be positioned outside of the patient's body and may itself not be exposed to the sudden change in the environment and/or may not otherwise be susceptible to the same effects from the environmental change. The solid-state sensors may be coupled to an elongate body (e.g., a catheter) that may be advanced into the patient's body, thereby exposing the solid-state sensors (e.g., sensors of the second sensor type) to the sudden change in the environment. The data from the first sensor type, such as a fluid-column based pressure sensor, may then be used to calibrate the data from the solid-state pressure sensors.

For example, in variations in which solid-state pressure sensors of a blood flow control device are calibrated, the elongate body may comprise an expandable member (e.g., balloon) and the sensor of the first sensor type positioned outside of the patient's body may be an expandable member sensor configured to measure one or more characteristics (e.g., pressure within the expandable member) of the expandable member. To calibrate the solid-state sensors, a fluid calibration bolus (referred to herein as a “calibration bolus”) may be injected into the expandable member, e.g., using a pump that may be fluidly coupled to the expandable member via a fluid column. In some variations, the calibration bolus may be saline. The volume of the calibration bolus may be at least equal to a desired volume of fluid referred to herein as a “non-disruptive volume.” The calibration process for the solid-state sensors may be initiated after at least the non-disruptive volume is injected into the expandable member. In some variations, the non-disruptive volume of fluid may be an amount of fluid that fills the fluid column with fluid but does not distend (or minimally distends) the expandable member. For example, the non-disruptive volume of fluid may be an amount of fluid that fills the fluid column with fluid but does not distend the expandable member to the extent that the expandable member may exert significant force on the fluid in the expandable member. Because the non-disruptive volume of fluid does not distend (or minimally distends) the expandable member, the pressure (e.g., blood pressure) outside the expandable member may be the same as the pressure within the expandable member. This pressure within the expandable member may be transduced to the expandable member sensor via the fluid column. When the volume of the calibration bolus is more than the non-disruptive volume but below an upper limit, the pressure within the expandable member may still be transduced to the expandable member sensor via the fluid column since the fluid column is filled with the fluid, thereby making it possible to calibrate one or more solid-state sensors. The sensor readings from the expandable member sensor may be used to the adjust the sensor readings from the solid-state sensors, thereby calibrating the solid-state sensors based on sensor data from the expandable member sensor.

Accordingly, systems, devices, and methods for calibrating solid-state sensors to provide accurate sensor data (e.g., pressure data), and especially accurate absolute sensor data (e.g., absolute pressure data instead of relative pressure data that may indicate relative pressure between two or more solid-state sensors), are described herein. In some variations, absolute pressure data may incorporate ambient pressure of the environment. In some variations, absolute pressure data may include pressure at a location (e.g., location of the solid-state sensor) with respect to the atmospheric pressure (e.g., gauge pressure). In particular, a method for calibrating one or more solid-state sensors coupled to an elongate body comprising an expandable member is described herein. In some variations, the method may include advancing the expandable member to a target location in a blood vessel of a patient, at which point a calibration bolus may be injected into the expandable member. A first sensor data, which may represent pressure in the expandable member, may be obtained using a controller from an expandable member sensor and a second sensor data may be obtained using a controller from the one or more solid-state sensors. The second sensor data may be adjusted based on the first sensor data, thereby calibrating the solid-state sensor utilizing data obtained from an expandable member sensor.

The systems, devices, and methods may also be configured to apply vacuum to the expandable member (e.g., a balloon). The vacuum may be applied before or during the process of calibrating the solid-state sensors, and may be used as a safeguard during calibration.

### System

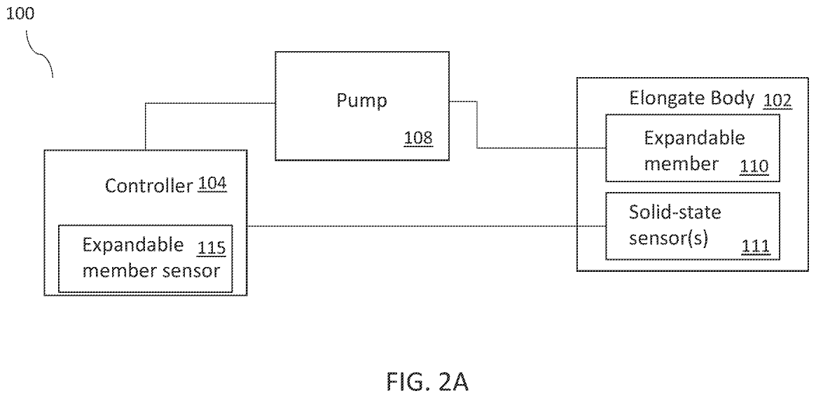

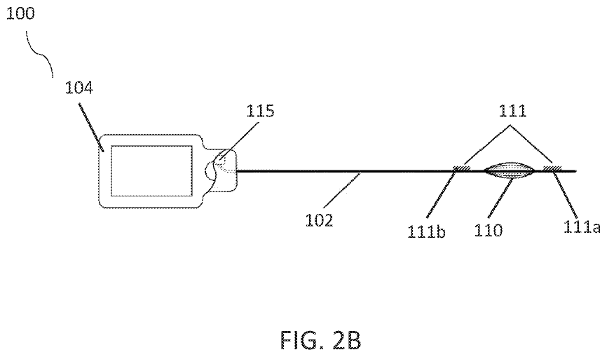

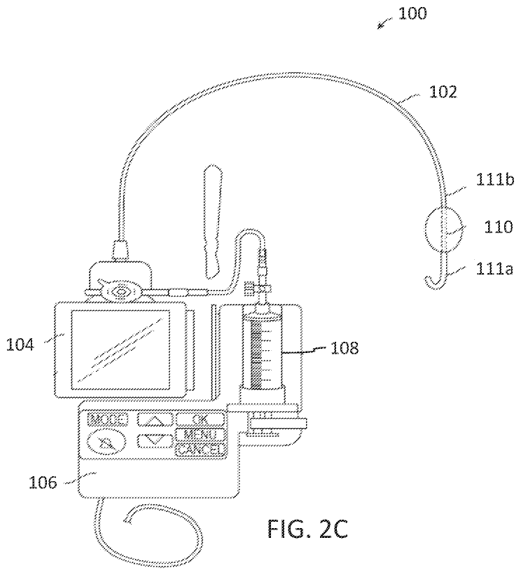

FIG. 2A is a block diagram illustrating an exemplary variation of a system 100 for measuring a physiological condition using solid-state sensors, which may additionally be used to calibrate the solid-state sensors. FIGS. 2B and 2C illustrate an exemplary variation of the system 100. The system 100 may include an elongate body 102 comprising an expandable member 110. An expandable member sensor 115 configured to measure one or more characteristics of (e.g., pressure within) the expandable member 110 may be positioned within a controller 104. For instance, the expandable member sensor 115 may be contained within, attached to, integrated with, or otherwise coupled to the controller 104. The expandable member 110 may be fluidly coupled via one or more fluid columns to the expandable member sensor 115. One or more solid-state sensors 111 may be coupled to the elongate body 102. For instance, one or more solid-state sensors 111 may be attached to, integrated with, and/or otherwise mounted on the elongate body 102. For example, the elongate body 102 may include a first solid-state sensor proximal to the expandable member 110 referred to as “distal solid-state sensor 111b” and a second solid-state sensor distal to the expandable member 110 referred to as “proximal solid-state sensor 111a”. In some variations, the system 100 may include more than one controller. For example, as seen in FIG. 2C, the system 100 may include a second controller 106 that may be operably coupled to the first controller 104. Controllers 104 and/or 106 may be communicatively coupled to the solid-state sensors 111 and/or the expandable member sensor 115 to receive and analyze sensor readings from the solid-state sensors 111 and/or the expandable member sensor 115. A pump 108 (e.g., a syringe pump) may be fluidly coupled to the expandable member 110 so as to adjust a volume of the expandable member 110. For example, the pump may be fluidly coupled via one or more fluid pathways (e.g., tubing, valves, lumen in the elongate body) to the expandable member 110 to inject fluid into and/or remove fluid from the expandable member 110.

### Elongate Body

The devices described herein may comprise an elongate body 102 comprising one or more sensors (e.g., one, two, three, four, five, or more), and in particular, the sensors may be integrated into the elongate body 102. The elongate body 102 may comprise a shaft sized and shaped for placement within a body of a patient (e.g., at a point of intervention such as in a blood vessel, parenchyma of the brain, esophagus, stomach, small intestine, etc.). In some variations, the elongate body 102 may be steerable. For example, in some variations, the elongate body 102 may be mechanically coupled to knobs, levers, pullwires, and/or the like that may be used to steer or otherwise deflect a distal end of the shaft of the elongate body 102. In some variations, the elongate body 102 may include one or more lumens therethrough. The lumen(s) may be partial lumen(s) (e.g., open on one end) and may be disposed within or lie within the shaft (e.g., steerable shaft). The one or more lumens (e.g., two, three, four, or more) may serve any desired purpose. For example, in some variations, the lumens may be used for transmitting fluids to and from a patient's body and/or other components coupled to the elongate body, advancing and/or steering a guidewire into a desired location, housing other components (e.g., sensor wires, pressure sensing columns, imaging devices such as endoscopes, etc.), etc. In some variations, the lumen(s) may include an intake lumen and an exhaust lumen to deliver fluid and/or compressed gas through the elongate body. In some variations, the elongate body 102 may include a lumen to fluidly couple the expandable member 110 to the pump 108. The lumen fluidly coupling the expandable member 110 to the pump 108 may also serve as the fluid column fluidly coupling the expandable member 110 to the expandable member sensor 115. Additionally or alternatively, the elongate body may include another fluid column to fluidly couple the expandable member 110 to the expandable member sensor 115. The pressure within the expandable member 110 may be transduced via the fluid column to the expandable member sensor 115.

As mentioned above, the elongate body 102 may be sized and shaped for advancement to and placement at least partially within a target location of the patient's body. The elongate body 102 may be any diameter and length suitable for advancement to the target location. For example, the elongate body 102 may have a diameter between about 2 mm and about 36 mm, including all values and sub-ranges therein. In some variations, the diameter may be for example between about 3 mm and about 25 mm, between about 4 mm and about 20 mm, or between about 5 mm and about 15 mm (including all values and sub-ranges therein). In some variations, the diameter may be for example between about 6 mm and about 10 mm. The elongate body 102 may have a length between about 1 cm and about 110 cm, including all values and sub-ranges therein. In some variations, the length may be for example between about 10 cm and about 105 cm, between about 20 cm and about 100 cm, between about 30 cm and about 90 cm, between about 40 cm and about 80 cm, or between about 50 cm and 70 cm (including all values and sub-ranges therein).

In some variations, the elongate body 102 may comprise multiple layers. For example, one or more portions of the elongate body 102 may comprise a plurality of layers (e.g., two, three, four, or more), and all portions of the elongate body may comprise the same layers, or the layers may differ between different portions of the elongate body 102. In other variations, the elongate body 102 may comprise a single layer that may include one or more lumens therethrough. The elongate body 102, and/or any layer of the elongate body, may be formed from any suitable biocompatible material, such as, for example, Polytetrafluoroethylene (PTFE), polyimide, and Pebax®, a combination thereof, and the like.

One or more sensors 111 (e.g., solid-state sensors) may be integrated into the elongate body 102. One or more sensor wires associated with the sensors 111 may be routed through the elongate body 102. In some variations, the elongate body 102 may comprise an opening and/or a window to receive the sensor(s) 111 via a sensor housing. The window and/or opening may receive the sensor(s) 111 via the sensor housing as an inset. In some variations, the window and/or opening may be a cavity formed with an outer layer of the elongate body 102 that may receive the sensor(s) 111 and/or the sensor housing. In some variations, the sensor(s) 111 may be positioned on or otherwise may contact an outer surface of the elongate body 102. In some variations, the sensors 111 may be integrated into the elongate body 102 in a manner similar to that described in International Application No. PCT/US2022/049335, the content of which is hereby incorporated by reference in its entirety.

In variations comprising a plurality of sensors 111, the sensors 111 may be positioned along and around the elongate body in any suitable manner. For example, two or more sensors may be longitudinally aligned along the elongate body. For example, a first sensor and a second sensor may be positioned along the same longitudinal line, but the first sensor may be positioned closer to a proximal end of the elongate body while the second sensor may be positioned closer to the distal end of the elongate body than the first sensor. In some variations, one or more sensors 111 may not be aligned longitudinally and may instead be circumferentially offset from one or more additional sensors such that the sensors 111 are located around the elongate body. In some variations, the circumferential offset (e.g., the angle formed between the longitudinal axes of the sensors) may be between 15 degrees and 345 degrees, such as, for example, 90 degrees, 180 degrees, or 270 degrees. In some variations, one or more sensors may be circumferentially offset from one or more additional sensors but may be aligned between the proximal and distal ends of the elongate body, while in other variations one or more sensors may be circumferentially offset from one or more additional sensors and may be positioned at different locations between the proximal and distal ends of the elongate body.

### Solid-State Sensor(s)

As mentioned above, the elongate body 102 may include one or more sensor(s). In some variations, one or more sensor(s) on the elongate body may be solid-state sensor(s) 111. The solid-state sensor(s) 111 may be attached to, integrated with, and/or otherwise mounted on the elongate body 102 in any suitable manner. As discussed above, the solid-state sensors 111 may be integrated into the elongate body 102 similar to the integration described in International Application No. PCT/US2022/049335, the content of which is hereby incorporated by reference in its entirety.

The sensors 111 may be pressure sensors configured to measure changes in blood pressure. In some variations, in addition to the pressure sensors, the sensors 111 may include sensors configured to measure other physiologic conditions such as heart rate, respiratory rate, intracranial pressure, cerebral oxygenation, cerebral blood flow, electroencephalography (EEG) signals, and the like. In some variations, the sensors 111 may include any sensor useful during a medical procedure such as, for example, temperature sensors, electrochemical sensors, impedance sensors, microelectrochemical system (MEMS) sensors, piezoelectric sensors, and/or the like. Any suitable number of sensors (e.g., one, two, three, four, or more) may be integrated into the elongate body to measure physiologic conditions.

In some embodiments, the devices may include two sensors, a first, distal sensor 111b, and a second, proximal sensor 111a, integrated into or otherwise coupled to the elongate body 102. In some variations, the distal sensor 111b may be integrated proximal to the expandable member 110 while the proximal sensor 111a may be integrated distal to the expandable member 110. For example, the distal sensor 111b located on the proximal side of the expandable member 110 may be placed at a distance from the expandable member 110 such that the physiologic data collected from the distal sensor 111b may not be disrupted by the blood flow downstream of the expandable member 110. In some variations, the distal sensor 111b may be placed at a distance between about 30 mm and about 10 mm, between about 25 mm and about 15 mm, between about 22 mm and about 18 mm from the expandable member 110. For instance, the distal sensor 111b may be placed approximately 20 mm from the expandable member 110. In some variations, the proximal sensor 111a located on the distal side of the expandable member 110 may be placed between about 30 mm and about 10 mm, between about 25 mm and about 15 mm, or between about 22 mm and about 18 mm from the expandable member 110. For instance, the proximal sensor 111a may be placed approximately 20 mm from the expandable member 110. In some variations, sensors 111 on the elongate body 102 may be situated at a specific distance from the ends of the expandable member 110 so as to acquire the physiologic data upstream and downstream of the expandable member 110.

Each of the distal sensor 111b and the proximal sensor 111a may measure patient physiologic information at a point of intervention to determine the patient's underlying physiology and to provide that information to a user. For example, in a variation in which the distal 111b and proximal 111a sensors may be blood pressure sensors, the distal sensor 111b and the proximal sensor 111a may measure a local blood pressure of the patient at or around the position of the respective sensor. The data from the distal sensor 111b may be used to measure the distal systolic pressure and the distal diastolic pressure of the patient. For instance, distal systolic pressure and distal diastolic pressure may be derived from a waveform of the blood pressure. Distal systolic pressure may be measured by analyzing peaks of the waveform for a given time duration. Distal diastolic pressure may be measured by analyzing valleys of the waveform for the given time duration. In a similar manner, the data from the proximal sensor 111a may be used to measure the proximal systolic pressure and the proximal diastolic pressure of the patient. For instance, proximal systolic pressure and proximal diastolic pressure may be derived from a waveform of the blood pressure. Proximal systolic pressure may be measured by analyzing peaks of the waveform for a given time duration. Proximal diastolic pressure may be measured by analyzing valleys of the waveform for the given time duration.

Note that the terms “proximal” and “distal,” as used herein in relation to sensor(s) and/or particular localized blood pressure readings, refer to blood flow directionality from the heart. That is, “proximal” is closer to the heart while “distal” is further from the heart. This is not to be confused with the reversed usage of the terms when described from the perspective of a medical device such as a catheter, where the “distal end” of the medical device would commonly be understood as the end with the expandable element 110 furthest from the controller 104 and the “proximal end” would be understood as the end closer to the operator.

The data from the sensor may be collected continuously or intermittently and may be collected over a defined period of time. In some variations, the data from the sensor may be collected continuously, such as for example, every 3 seconds, 4 seconds, 5 seconds, 6 seconds, 7 seconds, 8 seconds, 9 seconds, or 10 seconds (including all values and sub-ranges therein, such as, for example, between about 3 seconds and about 6 seconds, about 4 seconds and about 6 seconds, or between about 5 seconds or about 6 seconds). In some variations, the data from the sensor 118 may be collected every 5 seconds at 200 Hz.

### Expandable Member

The expandable member 110 may be disposed on, coupled to, integrated with, attached to, and/or affixed to the shaft of the elongate body 102 and a size (e.g., volume) of the expandable member may be controllable by a controller 104 or a user. For example, the expandable member may be configured to expand and contract and/or inflate and deflate such that the size (e.g., volume) of the expandable member may change during use of the blood flow control system. In some variations, the expandable member may be an inflatable/deflatable balloon, while in other variations the expandable member may comprise a shape memory material. In yet other variations, the expandable member may be connected to a mechanical linkage (e.g., wires, etc.) to change the size of the expandable member. The expandable member 110 may comprise any suitable elastomeric material (e.g., polyurethane, silicone, etc.). Additionally or alternatively, the expandable member 110 may comprise polyester, nylon, etc.

Blood flow at a target location in a patient's body (e.g., target blood vessel) may be regulated or otherwise controlled by changing a size of the expandable member 110. Fluid and/or compressed gas may be delivered through one or more lumens in the elongate body 102 in order to control and/or adjust the size (e.g., volume) of the expandable member 110. In some variations, the expandable member 110 may be strategically placed within the aorta of a patient and the size of the expandable member 110 may control blood flow through the aorta of the patient such that blood flow distal to expandable member 110 may be impeded to augment blood pressure proximal to expandable member 110. The outer surface of the expandable member 110 may be configured to contact or otherwise interface with the wall(s) of the patient's blood vessel (e.g., at complete occlusion). The expandable member 110 may have any suitable shape when inflated. In some variations, the expandable member 110 may have an oval cross-sectional shape along a longitudinal axis when inflated. In other variations, the expandable member 110 may have a spherical shape when inflated (e.g., may have a circular cross-sectional shape).

Although FIG. 2A illustrates a system 100 with a single expandable member 110, it should be readily understood that the elongate body 102 may include any number of suitable expandable members 110. For instance, the system 100 may include two, three, four, or more expandable members 110 disposed on, coupled to, integrated with, attached to, and/or affixed to the elongate body 102 in series. In variations comprising three or more expandable members, the distance between the expandable members may be the same, or it may be different. In some variations, the expandable members 110 may be balloons, which may be positioned in series along the length of the elongate body 102 or disposed within one another. In variations comprising a plurality of balloons, the balloons may be individually expanded and contracted, or they may be expanded and contracted together.

The expandable member 110 may be fluidly coupled to an expandable member sensor 115 that may be configured to detect a pressure inside the expandable member 110. For example, the expandable member 110 may be fluidly coupled to the expandable member sensor 110 via one or more fluid columns within the elongate body 102. The pressure inside the expandable member 110 may be transduced through the fluid columns to the expandable member sensor 115. In some variations, the controller 104 may include the expandable member sensor 115 and may be configured to analyze the pressure inside the expandable member 110 via sensor readings from the expandable member sensor 115.

### Pump

The system 100 may comprise a pump 108, such as a syringe pump, which may be operably (e.g., fluidly) coupled to the expandable member 110 to facilitate adjusting a size thereof. In some variations, the pump 108 may be contained within or otherwise carried by or coupled to the controller 104. Additionally or alternatively, the pump 108 may be communicably coupled to the controller 104. In some variations, the pump 108 may be operated manually (e.g., actuated by a user by hand, without use of the controller 104), or by using the controller 104. In some variations, the pump 108 may be operated automatically using the controller 104. Additionally or alternatively, the 108 may be operated via a user interface (e.g., buttons) on the controller 104. In some variations, the pump 108 may be detached from or otherwise decoupled from the controller 104, and may be operated manually so as to establish a position and initial level or volume of the expandable member 110.

The pump 108 may comprise or otherwise be coupled to the expandable member 110 comprising a lumen (e.g., tubing), which may in turn be coupled to a lumen of the elongate body 102 of the system 100. In this manner, the pump 108 may be in fluid communication with the expandable member 110.

In some variations, a set of one or more valves may be utilized to control the flow of a fluid, such as saline, and/or compressed gas, such as carbon dioxide. In some variations, the pump 108 may be fluidly coupled to a valve (e.g., a stopcock valve) that may regulate the flow of fluid and/or compressed gas to the expandable member 110.

The size (e.g., volume) of the expandable member 110 may be adjusted using the controller 104 and the pump 108. For example, the controller 104 may determine an amount of fluid and/or compressed gas that is to be injected into or removed from the expandable member 110 so as to adjust the size of the expandable member 110 and thereby affect blood flow. The controller 104 may control (e.g., move, modify, or control a position thereof) an actuator, which may be releasably coupled to the pump 108 (e.g., to an actuation element on the pump). The actuator may engage and move the actuation element, thereby moving a portion of the pump 108 such that the pump 108 may inject or remove the fluid and/or compressed gas from the expandable member 110 based on instructions from the controller 104. In some variations, removal of the fluid and/or compressed gas may be activated via a screw actuation. In some variations, the pump may be coupled to a position sensor, which may provide information on the position of a portion of the pump 108 and thus how much fluid has been delivered to the expandable member 110.

The pump 108 may be any suitable pump operably and/or communicatively coupled to an actuator so as to inject and/or remove the fluid and/or compressed gas from the expandable member 110. For example, the pump 108 may be a syringe pump, diaphragm pump, peristaltic pump, or other suitable pump. In some variations, the system may comprise a plurality of pumps. For example, a first pump may be used to inject a calibration bolus into the expandable member, and a second, different pump may be used to meter fluid into and out of the expandable member to control blood flow. A third pump, different from the first and second pumps, may be used to apply negative pressure to the expandable member, as discussed in more detail herein. In other variations, the same pump may be used to perform a plurality of functions, including injecting the calibration bolus, metering fluid into and out of the expandable member, and applying negative pressure to the expandable member.

### Expandable Member Sensor

The expandable member sensor 115 may be disposed on, affixed to, attached to, mounted on, coupled to, and/or otherwise contained within the controller 104. The expandable member sensor 115 may be configured to detect a characteristic of the expandable member 110 such as, for example, a pressure of fluid and/or compressed gas inside the expandable member 110. For example, the expandable member sensor 115 may be fluidly coupled to the expandable member 110 via one or more fluid columns (e.g., lumens within the elongate body, tubing)), such as, for example, through the elongate body 102. The pressure in the expandable member 110 may be transduced via the fluid pathways to the expandable member sensor 115 positioned and/or included in the controller 104. In some variations, the expandable member sensor 115 may measure the expandable member pressure. When the expandable member 110 is not distended, the expandable member pressure may be indicative of the pressure outside of and surrounding the expandable member 110. When the expandable member is inflated, the expandable member pressure may be indicative of an amount of inflation of the expandable member 110. The expandable member pressure may be indicative of the amount of inflation and deflation of the expandable member 110.

In some variations, data may be collected from the expandable member sensor 115 continuously such as, for example, every 3 milliseconds, 4 milliseconds, 5 milliseconds, 6 milliseconds, 7 milliseconds, 8 milliseconds, 9 milliseconds, or 10 milliseconds (including all values and sub-ranges therein, such as, for example, between about 3 milliseconds and about 6 milliseconds, about 4 milliseconds and about 6 milliseconds, or between about 5 milliseconds and about 6 milliseconds).

In some variations, and as will be described in more detail herein, sensor readings from the expandable member sensor 115 may be used to calibrate the solid-state sensors 111. More specifically, expandable member pressure obtained from the expandable member sensor 115 when the expandable member 110 is not distended may be used to calibrate the solid-state sensors. For example, one or more pressure waveforms indicative of pressure within the expandable member 110 obtained from the expandable member sensor 115 may be compared to one or more pressure waveforms obtained from the solid-state sensors 111. The solid-state sensors 111 may be calibrated by adjusting the pressure waveforms from the solid-state sensors 111 based on the comparison with the pressure waveforms obtained from the expandable member sensor 115. For instance, if the pressure waveform from the expandable member sensor 115 includes an increasing slope for a first time duration, the pressure waveform from the solid-state sensors 111 may be adjusted to have a similar increasing slope for the first time duration. In particular, a change in gradient in the pressure waveform from the solid-state sensors 111 may be adjusted to be the same as and/or substantially similar to (e.g., within ±10 mmHg) a change in gradient in the pressure waveform from the expandable member sensor 115. For example, an increase in slope of the pressure waveform obtained from the solid-state sensors 111 for the first duration may be adjusted to match (e.g., be the same as) a rate of increase in slope of the pressure waveform obtained from the expandable member sensor 115 for the first duration. Similarly, if the pressure waveform from the expandable member sensor 115 includes a decreasing slope for a second time duration, the pressure waveform from the solid-state sensors 111 may be adjusted to have a similar or the same decreasing slope for the second time duration.

In some variations, the expandable member sensor 115 may be a gauge pressure sensor. In some variations, the expandable member sensor 115, while factory calibrated, may still be sensitive to atmospheric pressure and/or altitude. That is, the atmospheric pressure and/or altitude at a location may influence the sensor data obtained from the expandable member sensor 115. Accordingly, prior to advancing the system 100 into the patient's body, an initial setpoint may be determined for the expandable member sensor 115 to account for the impact of the atmospheric pressure and/or altitude at the location where the medical procedure is being performed as further described below. Additionally or alternatively, a barometric sensor may be included in the controller (104 or 106), which can be used to compensate for changes in barometric pressure and or altitude.

### Controller

The devices and/or systems described herein may comprise one or more controllers (e.g., controller 104 and controller 106). For example, the system 100 may comprise a first controller 104, which may be coupled to a base of the elongate body 102. The first controller 104 may be operably coupled to the solid-state sensors 111 and/or the expandable member sensor 115. In some variations, a second controller 106 may be releasably coupled to the first controller 104 as shown in FIG. 2C. In such variations, the second controller 106 may be coupled to the elongate body 102 via the first controller 104. In some variations, the system 100 may not include the first controller 104 and the second controller 106 may be directly coupled to the elongate body 102.

The controller(s) (e.g., controller 104 and/or controller 106) may be communicatively coupled to the sensors, such as, for example, the sensors integrated into the elongate body and may receive data therefrom. The controller(s) may comprise a processor (e.g., CPU) that may process data and/or other signals to control one or more components of the system. The processor may be configured to receive, process, compile, compute, store, access, read, write, and/or transmit data and/or other signals. In some variations, the processor may be configured to access or receive data and/or other signals from one or more of a sensor and a storage medium (e.g., memory, flash drive, memory card). The processor may be configured to run and/or execute application processes and/or other modules, processes and/or functions associated with the device.

In some variations, data from the sensors may be analyzed at the controller(s) over a discrete period of time. For instance, the data may be analyzed for example, every 3 milliseconds, 4 milliseconds, 5 milliseconds, 6 milliseconds, 7 milliseconds, 8 milliseconds, 9 milliseconds, or 10 milliseconds (including all values and sub-ranges therein, such as, for example, between about 3 milliseconds and about 6 milliseconds, about 4 milliseconds and about 6 milliseconds, or between about 5 milliseconds or about 6 milliseconds).

In some variations, the controller(s) may be communicably coupled to a user interface. For example, the user interface may be a display on the controller(s). In some variations, the user interface may be a display on any suitable computing device (e.g., computer, smartphone, tablets, etc.) communicably coupled to the controller, via, e.g., the communication device or module described herein. The user interface may comprise an input device (e.g., touch screen) and output device (e.g., display device) and be configured to receive input data from the sensor. In some variations, an input device may comprise a touch surface for an operator to provide input (e.g., finger contact to the touch surface) corresponding to a control signal. In some variations, a haptic device may be incorporated into one or more of the input and output devices to provide additional sensory output (e.g., force feedback) to the operator.

The controller 104 and/or controller 106 may be configured to calibrate the solid-state sensors 111 based on sensor readings from the expandable member sensor 115 as further described herein. For example, the controller 104 may be configured to calibrate the solid-state sensors 111. Additionally or alternatively, the controller 106 may be configured to calibrate the solid-state sensors 111. In some variations, the entire calibration process may be performed by either of the controllers 104, 106 alone. In some variations, the calibration process may be split between the controllers 104,106. The following paragraphs discuss calibration using controller 104. It should be readily apparent that one or more of these steps may be performed additionally or alternatively by controller 106.

In some variations, the controller 104 may automatically calibrate the solid-state sensors 111 based on the sensor readings from the expandable member sensor 115. In some variations, the controller 104 may partially calibrate the solid-state sensors 111 automatically and partially calibrate the solid-state sensors 111 based on feedback from a user (e.g., surgeon, operator, doctor, etc.). For example, the controller 104 may initiate a calibration process (e.g., calibration process to calibrate the solid-state sensors) in response to and/or based on instructions and/or notification from the user (e.g., feedback from the user). However, in some variations, the controller 104 may analyze sensor readings from the expandable member sensor 115 automatically to calibrate the solid-state sensors 111.

The solid-state sensors 111 and the expandable member sensor 115 may be initially calibrated (e.g., factory calibrated) prior to being communicatively and/or operably coupled to the controller 104. For example, a calibration curve and/or a calibration constant may be determined and/or set for each of the expandable member sensor 115 and the solid-state sensors 111 before the sensors are coupled to the controller 104. More specifically, since sensors convert a measurement of a physical quantity (e.g., temperature, pressure, strain, optical energy, etc.) into electrical signals that may analyzed, the calibration curve may represent the relationship between the physical quantity the sensor is configured to measure and the respective sensor output, such as voltage. For example, the calibration curve may indicate the relationship between pressure measurements and the corresponding voltage outputs. When the sensors are in use, voltage output from the sensors may be analyzed by the controller 104 to determine a change in pressure as sensed and measured by each of the sensors. In some variations, the calibration curve may be linear. As an example, the calibration curve may be a straight line, such as y=mx+c, where y may indicate the output voltage and x may indicate a change in pressure as measured and sensed by the sensors, and c may indicate a calibration constant.

In some variations, after the initial calibration (e.g., factory calibration), the solid-state sensors 111 and/or the expandable member sensor 115 may be coupled to, attached to, or otherwise integrated with the system 100. In other variations, the initial calibration described above may be performed after the solid-state sensors 111 and/or the expandable member 115 are coupled to, attached to, or otherwise integrated with the system 100. Prior to using the system 100 to monitor physiologic conditions in a patient, the controller 104 may determine an initial setpoint for the expandable member sensor 115. The expandable member sensor 115 may be sensitive to environmental factors such as, for example, atmospheric pressure and/or altitude. For instance, a change in the atmospheric pressure and/or altitude may influence the sensor readings. Accordingly, the controller 104 may determine an initial setpoint for the expandable member sensor 115 that may account for the influence that the environmental factors may have on the sensor data. In some variations, the controller 104 may include one or more controls (e.g., user interface with touchscreen buttons, physical buttons, etc.) that the user can manipulate (e.g., press, push, pull, etc.) to indicate to the controller to determine a baseline value, such as a zero point for the expandable member sensor 115. Additionally or alternatively, the controller 104 may automatically determine a baseline value for the expandable member sensor 115. For example, the measurement of pressure and/or sensor readings indicative of pressure from the expandable member sensor 115 may be set to zero (even if the measurement and/or reading from the expandable member sensor 115 does not show zero) prior to advancing the elongate body 102 into the patient's body. Additionally or alternatively, a barometer may be communicatively and/or operably coupled to the controller 104. The controller 104 may receive sensor data from the barometer that may be indicative of the atmospheric pressure (e.g., every second, every 5 milliseconds, etc.). The atmospheric pressure may be used to determine an initial setpoint for the expandable member sensor 115. For example, the atmospheric pressure received from the barometer may be set as the zero point for the expandable member sensor 115.

A user (e.g., surgeon, operator, doctor, etc.) may advance at least a portion of the system 100 into a target location in a patient's body. For example, the user may advance at least the expandable member 110 to a target location in a patient's body. When the solid-state sensors 111 transition from air outside of the patient's body to fluid inside the patient's body, the controller 104 may be configured to identify this transition. For instance, the controller 104 may analyze sensor data from the solid-state sensors 111 to identify a rapid change in sensor readings. For example, the controller 104 may identify a rapid drop or decrease and/or a rapid rise or increase in sensor data that may be indicative of a transition between media. For instance, after the portion of the system is advanced into the target location in a patient's body, a sudden change in gradient in the pressure waveform (e.g., arterial waveforms, venous waveforms, etc.) obtained from the solid-state sensors 111 may indicate that the transition from air outside of the patient's body to fluid inside of the patient's body has occurred. The controller may identify this sudden change in gradient and may optionally notify the user that the transition has occurred. The change in gradient (e.g., rapid rise and/or rapid drop) may range from about 15 mmHg/sec to about 1200 mmHg/sec, including all values and sub-ranges therein. For example, the change in gradient may be about 15 mmHg/sec, about 25 mmHg/sec, about 50 mmHg/sec, about 100 mmHg/sec, about 150 mmHg/sec, about 200 mmHg/sec, about 250 mmHg/sec, about 300 mmHg/sec, about 350 mmHg/sec, about 400 mmHg/sec, about 450 mmHg/sec, about 500 mmHg/sec, about 550 mmHg/sec, about 600 mmHg/sec, about 650 mmHg/sec, about 700 mmHg/sec, about 750 mmHg/sec, about 800 mmHg/sec, about 850 mmHg/sec, about 900 mmHg/sec, about 950 mmHg/sec, about 1000 mmHg/sec, about 1050 mmHg/sec, about 1100 mmHg/sec, about 1150 mmHg/sec, or about 1200 mmHg/sec. Additionally or alternatively, the controller 104 may include one or more controls (e.g., user interface with touchscreen buttons, physical buttons, etc.) that the user can manipulate (e.g., press, push, pull, etc.) to indicate the transition.

As discussed herein, this rapid change in sensor data may yield inaccurate sensor readings. For example, the solid-state sensors 111 may be configured to measure physiologic conditions such as blood pressure in the target location. A sudden change in gradient in the pressure waveform due to the transition of media may yield inaccurate blood pressure measurements in the target location. Accordingly, it may be advantageous to calibrate the solid-state sensors 111 after the sudden change so that the sensor data obtained from the solid-state sensors 111 indicate the actual blood pressure at the target location. To do so, the calibration curve and/or the calibration constant for the solid-state sensors 111 may be adjusted based on the sensor data from the expandable member sensor 115 as further described herein.

An inherent offset may exist between the sensor data from the proximal solid-state sensor 111a and the distal solid-state sensor 111b after advancement of the expandable member 110 to the target location and before delivery of any fluid to the expandable member. In some variations, this inherent offset may be due to the presence of the expandable member 110 positioned between the proximal solid-state sensor 111a and the distal solid-state sensor 111b, which may create a pressure difference between the proximal solid-state sensor 111a and the distal solid-state sensor 111b. Additionally or alternatively, this inherent offset between the proximal solid-state sensor 111a and the distal solid-state sensor 111b may be due to the rapid change in sensor data that results from the transition from one medium to another (e.g., from air outside the patient's body to fluid inside the patient's body) and/or the inherent offset may be an inherent difference between two specific sensors (despite the proximal solid-state sensor 111a and the distal solid-state sensor 111b being the same type of sensor).

Accordingly, after advancing the expandable member 110 to the target location (e.g., target location in a blood vessel) and before injecting fluid into the expandable member 110, the controller 104 may determine the inherent offset between the proximal solid-state sensor 111a and the distal solid-state sensor 111b. Because fluid has not yet been injected into the expandable member 110, the expandable member 110 may not affect the blood flow at the target location or may not materially and/or substantially affect the blood flow (e.g., due to the difference in pressures measured by the proximal solid-state sensor and the distal solid-state sensor being not more than about 1.0 mmHg) at the target location, and thus the sensor data from the proximal solid-state sensor and the distal solid-state sensor 111b should be the same. However, the data between the two solid-state sensors may be different due to the inherent offset.

For example, consider sensor readings obtained from (i.e., pressure measured at) the proximal solid-state sensor 111a and the distal solid-state sensor 111b. As discussed herein, the proximal solid-state sensor 111a may be positioned distal to the expandable member 110. Accordingly, the proximal solid-state sensor 111a may also be referred to herein as a “tip sensor.” The pressure measured at the proximal solid-state sensor 111a (tip sensor) may be represented as T_{measured}. Similarly, the distal solid-state sensor 111b may be positioned proximal to the expandable member 110. Accordingly, the distal solid-state sensor 111b may be referred to as a “hub sensor.” The pressure measured at the distal solid-state sensor 111b (hub sensor) may be represented as H_{measured}. However, the actual pressure at the proximal solid-state sensor may be represented as T_{actual} and the actual pressure at the distal solid-state sensor 111b may be represented as H_{actual}. Therefore, the pressure measured (T_{measured}) at the proximal solid-state sensor 111a may be the actual blood pressure (T_{actual}) at the proximal solid-state sensor 111a plus a proximal solid-state sensor offset represented as ∈_{T}. Similarly, the pressure measured (H_{measured}) at the distal solid-state sensor 111b may be the actual blood pressure (H_{actual}) at the distal solid-state sensor 111b plus a distal solid-state sensor offset represented as ∈_{H}.

T_{measured}=T_{actual}+∈_{T} (1)

H_{measured}=H_{actual}+∈_{H} (2)

As discussed herein, before the expandable member 110 begins to materially and/or substantially disrupt the blood flow (e.g., affect the pressures measured by the proximal solid-state sensor and the distal solid-state sensor by more than about 1.0 mmHg) at the target location, T_{measured} should be the same as H_{measured}. Put differently, consider that the pressure in the expandable member 110 is B.

When B<0, T_{actual}=H_{actual} (3)

However, the pressure measured at the proximal solid-state sensor 111a may not be the same as the pressure measured at the distal solid-state sensor 111b, and thus the inherent offset between the proximal solid-state sensor 111a and the distal solid-state sensor 111b may be determined by the controller 104 as:

α=T_{measured}−H_{measured}=∈_{T}−∈_{H} (4)

where α represents the inherent offset between the proximal solid-state sensor 111a and the distal solid-state sensor 111b.

After the inherent offset is determined, to calibrate the solid-state sensors, the expandable member 110 may be injected with a calibration bolus. In some variations, the volume of the calibration bolus may be equal to a non-disruptive volume of fluid, while in other variations the volume of the calibration bolus may be more than the non-disruptive volume of fluid, but below an upper volume limit. The non-disruptive volume of fluid may be an amount of fluid that does not distend the expandable member 110, but does fill the fluid column between the expandable member 110 and the pump 108. At the non-disruptive volume, the expandable member 110 may not materially and/or substantially affect the blood flow (e.g., due to the difference in pressures measured by the proximal solid-state sensor and the distal solid-state sensor being not more than about 1.0 mmHg) at the target location, however, since the fluid column is completely filled, the pressure from inside the expandable member 110 may be transduced to the expandable member sensor 115. More specifically, when the non-disruptive volume of fluid is added, the pressure from outside the expandable member 110 may start transducing to the expandable member sensor 115. Put another way, at the non-disruptive volume, the pressure outside the expandable member 110 at the target location may be the same as the pressure inside the expandable member 110. In this manner, the expandable member pressure may be representative of the blood pressure at the target location. It should be appreciated that the non-disruptive volume may refer to a single volume, or a range of volumes, and thus, when described herein as a volume greater than the non-disruptive volume, this may refer to a volume outside of, and above, the range of volumes.

In some variations, the volume of the calibration bolus may be a volume greater than the non-disruptive volume, but below an upper volume limit. For example, the volume of the calibration bolus may be greater than the non-disruptive volume such that the expandable member 110 may begin to become distended and the blood flow at the target location may be affected by the expandable member. However, the volume of the calibration bolus may not exceed an upper volume limit. The upper volume limit may be a volume at which the pressure inside the expandable member 110 is not the same as or is not substantially similar to (e.g., not within ±5.0 mmHg) the pressure outside the expandable member 110 at the target location, such as pressure at the proximal solid-state sensor. Put another way, at and above the upper volume limit, the pressure data received from the expandable member sensor may no longer be representative of the pressure outside the expandable member at the target location.

Accordingly, the volume of the calibration bolus may be such that the volume is at least the non-disruptive volume at which the pressure inside the expandable member 110 may start transducing to the expandable member sensor 115 but less than a volume at which the pressure inside the expandable member may not be the same or may not be substantially similar to (e.g., not within +5.0 mmHg) the pressure outside the expandable member 110. For example, the volume of the calibration bolus may be at least the non-disruptive value at which the pressure inside the expandable member may be γ_{lower}. Therefore, to calibrate the solid-state sensors 111, the expandable member pressure B should at least be a pressure γ_{lower}.

As mentioned above, the volume of the calibration bolus may be more than the non-disruptive volume. However, the volume should be less than the upper volume limit at which the pressure inside the expandable member is not the same or is not substantially similar to (e.g., not within ±5.0 mmHg) the pressure outside the expandable member 110. For example, the volume of the calibration bolus may not exceed a volume at which a pressure γᵤₚₚₑᵣ inside the expandable member 110 is not same as the pressure outside the expandable member 110. Therefore, to calibrate the solid-state sensors 111, the expandable member pressure B should be lower than γᵤₚₚₑᵣ.

Therefore, the set of possible expandable member pressure values B can be defined as:

∀B: B=balloon pressure (5)

B_{lowvol}={B:γ_{lower}<B and B<γᵤₚₚₑᵣ and γ_{lower}>0} (6)

In some variations, the controller 104 may be configured to verify whether the pressure in the expandable member 110 is between γ_{lower} and γᵤₚₚₑᵣ.

As discussed above, when the volume of the calibration bolus exceeds the non-disruptive volume but remains below the upper volume limit such that calibration is still possible, the expandable member 110 may begin to materially and/or substantially affect the blood flow (e.g., affect the pressures measured by the proximal solid-state sensor and the distal solid-state sensor by more than about 1.0 mmHg) at the target location. In such a scenario, the sensor data from the distal solid-state sensor 111b may begin to drop. For example, when the volume of the calibration bolus exceeds the non-disruptive volume, a gradient in the pressure waveform from the distal solid-state sensor 111b may decrease. However, a gradient in pressure waveform from the proximal solid-state sensor 111a may increase and/or remain unchanged. More specifically, a change in gradient in the pressure waveform from the distal solid-state sensor 111b may be different from a change in gradient in the pressure waveform from the proximal solid-state sensor 111a. In such a scenario, a post inflation offset between the distal solid-state sensor 111b and the proximal solid-state sensor 111a may be determined. For example, when a volume greater than the non-disruptive volume is injected into the expandable member 110, the pressure disruption due to the expandable member 110 affecting the blood flow may be represented as Ψ. The post inflation offset β between the proximal solid-state sensor 111a and the distal solid-state sensor 111b may be determined as:

β=Ψ+α (7)

where α represents the inherent offset between the proximal solid-state sensor 111a and the distal solid-state sensor 111b determined at Equation 4.

In some variations, the non-disruptive volume and/or the volume of the calibration bolus (to the extent different) may be determined by the controller 104. Additionally or alternatively, the non-disruptive volume and/or the volume of the calibration bolus may be preprogrammed into the controller 104. The non-disruptive volume may be determined based on one or more of: shape of the expandable member 110, volume of the expandable member 110, length of the elongate body 102, volume of the fluid column that fluidly couples and/or connects the expandable member 110 with the pump 108, length of fluid column coupling the expandable member 110 with the pump 102, amount of fluid needed to transition negative pressure readings obtained from the expandable member sensor 115 to zero pressure reading and/or a positive pressure reading, size of the blood vessel (e.g., size of the target location in the blood vessel), and/or a combination thereof. The volume of the calibration bolus may be equal to, or determined based on, the non-disruptive volume.

In some variations, the non-disruptive volume may be determined based, at least in part, on the shape of the expandable member 110. In some variations, the controller 104 may determine the non-disruptive volume based, at least in part, on the shape of the expandable member 110. For instance, the non-disruptive volume may be determined based on whether the expandable member 110 has an oval cross-sectional shape, circular cross-sectional shape, etc. when inflated.

In some variations, the non-disruptive volume may be determined based, at least in part, on the overall volume of the expandable member 110. For example, the controller 104 may determine the non-disruptive volume based, at least in part, on the overall volume of the expandable member 110. For example, in some variations, the non-disruptive volume may be between about 2% and about 5%, between about 2.5% and about 5%, between about 3% and about 5%, between about 3.5% and about 5%, between about 4% and about 5%, or between about 4.5% and about 5% of the total volume of the expandable member 110 (including all values and sub-ranges therein). In some variations, the non-disruptive volume may be about or less than about 5%, about or less than about 4.5%, about or less than about 4.2%, about or less than about 4%, about or less than about 3.7%, about or less than about 3.5%, about or less than about 3%, or about or less than about 2.5% of the volume of the expandable member 110.

In some variations, the non-disruptive volume may be determined based, at least in part, on one or more characteristics (e.g., length, volume of one or more lumens, etc.) of the elongate body 102. For example, the non-disruptive volume may be determined based on a volume of the fluid column within the elongate member 102 that fluidly couples the pump 108 (e.g., syringe pump) to the expandable member 110. As another example, the non-disruptive volume may be determined based on a length of the fluid column within the elongate member 102 that fluidly couples the pump 108 to the expandable member.

In some variations, the non-disruptive volume may be determined based, at least in part, on the amount of fluid needed to transition negative pressure readings obtained from the expandable member sensor 115 to zero pressure reading and/or a positive pressure reading. For example, when the expandable member 110 is advanced into the target blood vessel, a partial vacuum may be created in the expandable member 110. Therefore, a pressure differential may exist between the expandable member sensor 115 and the expandable member 110. Accordingly, the expandable member 110 may initially yield negative sensor readings (e.g., negative pressure readings) indicating the partial vacuum in the expandable member 115. Injecting the non-disruptive volume may overcome and/or eliminate the partial vacuum. Therefore, the non-disruptive volume be an amount that may yield a zero pressure reading and/or a positive pressure reading from the expandable member sensor 115, thereby eliminating or overcoming the partial vacuum in the expandable member 110.

In some variations, the volume of the calibration bolus may be determined based, at least in part, on the size of the bodily lumen (e.g., blood vessel) in which the expandable member 110 is advanced. For instance, the calibration bolus may be determined based on the size of the aorta or a vein the expandable member 110 is advanced into.

In some variations, the non-disruptive volume may be between 100 μl and 300 ml. In some variations, the non-disruptive volume may be between about 1 ml to about 1.5 ml. In some variations, the controller 104 may notify the user to inject the calibration bolus in the expandable member 110. In some variations, the controller 104 may include a user interface or a display to display the calibration bolus and/or the time point at which the calibration bolus is to be injected into the expandable member 110.

In some variations, a time point at which the calibration bolus is to be injected into the expandable member 110 may be determined. For example, the controller 104 may determine a time point at which the calibration bolus is to be injected into the expandable member 110. Additionally or alternatively, the time point at which the calibration bolus is to be injected may be preprogrammed into the controller 104. In some variations, the time point may be determined based on one or more of length of the elongate member, distance of the solid-state sensors 111 from the distal end of the elongate member, length of the fluid column that fluidly couples and/or connects the expandable member with the pump 108, a combination thereof, and/or the like. In some variations, the time point may be determined based on sensor data from the proximal solid-state sensor 111a and the distal solid-state sensor 111b. For example, the time point may be a time at or a time after a change in gradient of a pressure waveform from the proximal solid-state sensor 111a matches a change in gradient of a pressure waveform from the distal-solid state sensor 111b. As another example, the time point may be a time at or a time after pressure reading from the proximal solid-state sensor is the same as the pressure reading from the distal solid-state sensor

During or after the calibration bolus is injected into the expandable member, the controller 104 may be configured to determine whether the volume of the calibration bolus injected into the expandable member 110 is equal to the non-disruptive volume or whether the volume of the calibration bolus injected into the expandable member 110 is greater than the non-disruptive volume.

When the volume of the calibration bolus equals the non-disruptive volume, the controller 104 may further be configured to determine whether the non-disruptive volume has been injected or otherwise delivered to the expandable member. In some variations, the controller 104 may be further configured to transmit a notification indicating that the non-disruptive volume has been added when or after it has made the determination that the non-disruptive volume has been added. While described above in relation to the non-disruptive volume, it should be appreciated that the controller 104 may be configured to transmit a notification that the calibration bolus has been added.

The controller 104 may analyze sensor data from the solid-state sensors 111 and/or the expandable member sensor 115 in one or more of a variety of ways to determine whether the non-disruptive volume has been delivered. For example, the controller 104 may analyze the sensor data from the expandable member sensor 115 to determine whether the expandable member sensor data is zero or a positive value (e.g., positive pressure reading). If the controller determines that the expandable member sensor data is zero or a positive value, then the controller 104 may determine that the non-disruptive volume has been injected, and may optionally notify the user. Additionally or alternatively, if the controller 104 determines that the expandable member sensor data is a negative value, then the controller 104 may determine that the amount of fluid injected into the expandable member 110 is less than the non-disruptive volume. In some variations, the controller 104 may notify the user, e.g., via the user interface, that the amount of delivered fluid is less than the non-disruptive volume. In another example, the controller 104 may compare the sensor data from the proximal solid-state sensor 111a with the sensor data from the distal solid-state sensor 111b. If a change in gradient in the pressure waveform (e.g., sensor data) obtained from the proximal state sensor 111a is same as a change in gradient in the pressure waveform obtained from the distal state sensor 111b, the controller 104 may determine that the non-disruptive volume has been injected, and may optionally so notify the user. More specifically, the controller may determine whether the rise or drop in pressure measured at the proximal solid-state sensor 111a is the same as the rise or drop in pressure measured at the distal solid-state sensor 111b. If the rise or drop in pressure measured at the two pressure sensors is the same, then the controller 104 may determine that the non-disruptive volume has been added and may optionally so notify the user. As yet another example, the controller 104 may compare the sensor data received from the expandable member sensor 115 to the sensor data received from one or more of the solid-state sensors 111. If a change in gradient in the pressure waveform obtained from the expandable member sensor 115 is same as a change in gradient in the pressure waveform from one or more solid state sensors 111, the controller 104 may determine that the non-disruptive volume has been injected, and may optionally notify the user. More specifically, the controller 104 may determine whether the rise or drop in pressure measured from the expandable member sensor 115 is the same as the rise or drop in pressure measured at the one or more solid-state sensors 111. If the rise or drop in pressure measured from the expandable member sensor 115 and the one or more solid state sensors 111 is the same, then the controller 104 may determine that the non-disruptive volume has been added and may optionally so notify the user. It should be appreciated that the controller 104 described herein may utilize any or all of the above techniques to determine whether the non-disruptive volume has been added to, injected into, or otherwise delivered to the expandable member 110.

Additionally or alternatively, the controller 104 may be configured to determine whether the volume of the calibration bolus injected into the expandable member 110 is greater than the non-disruptive volume. For example, in one variation, the controller 104 may compare the sensor data received from the proximal solid-state sensor 111a and from the distal solid-state sensor 111b. If a difference begins to exist between the sensor data from the solid-state sensors 111a, 111b, then the controller 104 may determine that a volume above the non-disruptive volume has been added to the expandable member 110, and may optionally notify the user. For instance, if a change in gradient in the pressure waveform received from the proximal solid-state sensor 111a is different from a change in gradient in the pressure waveform received from the distal solid-state sensor 111b, then the controller 104 may determine that a volume greater than the non-disruptive volume has been added to the expandable member 110, and optionally so notify the user. More specifically, the controller 104 may determine whether the rise or drop in pressure measured at the proximal solid-state sensor 111a is different from the rise or drop in pressure measured at the distal solid-state sensor 111b. For instance, if there is a drop in pressure measured at the proximal solid-state sensor 111a and no drop in pressure measured at the distal solid-state sensor 111b, then the controller 104 may determine that a volume greater than the non-disruptive volume has been added to the expandable member 110 and may optionally notify the user. When the controller 104 determines that a volume greater than the non-disruptive volume has been injected into the expandable member, the controller 104 may perform one of two actions. In some variations, the controller 104 may calibrate the distal solid-state sensor 111b based on the sensor data from the proximal solid-state sensor 111a as further described herein. In other variations, the controller 104 may stop and/or terminate the calibration process and may optionally so notify the user.

Prior to calibrating the solid-state sensors 111, the controller 104 may determine whether a predetermined amount of time has elapsed from advancing the elongate body 102 into the target location in the patient's body. The calibration bolus may be injected into the expandable member 110 before the predetermined amount of time has elapsed. In some variations, the predetermined time may be between about 45 seconds and about 5 seconds, between about 30 seconds and about 5 seconds, between about 25 seconds and about 5 seconds, between about 15 seconds and about 5 seconds, between about 10 seconds and about 5 seconds (including all values and subranges therein) from when the expandable member 110 has been advanced into the target location. In some variations, the predetermined time may be preprogrammed into the controller 104 and the controller 104 may optionally notify the user once the predetermined time has elapsed. Additionally or alternatively, the user may manipulate one or more controls on the controller 104 to indicate that the predetermined time has elapsed.

As discussed above, the controller 104 may indicate that the calibration bolus has been added. Additionally or alternatively, the user may manipulate one or more controls on the controller 104 to indicate that the calibration bolus has been added. For example, the controller 104 may include a display indicating the volume of the fluid that has been injected into the expandable member 110. The user may manipulate (e.g., push, pull, press, etc.) the controls (e.g., user interface with touchscreen buttons, physical buttons, etc.) on the controller 104 to indicate that the calibration bolus has been added. At this point, the solid-state sensors 111 may be calibrated, as will be discussed in more detail herein.

To calibrate the solid-state sensors 111, the controller 104 may analyze the sensor data from the expandable member sensor 115. For example, to calibrate the proximal solid-state sensor 111a, controller 104 may analyze the sensor data from the expandable member sensor 115 and the proximal solid-state sensor 111a. The controller 104 may shift and/or adjust a calibration curve and/or a calibration constant of the proximal solid-state sensor 111a such that the sensor data from the proximal solid-state sensor 111a is the same as the sensor data from the expandable member sensor 115.

For example, when the volume of the calibration bolus equals the non-disruptive volume, the expandable member 110 does not significantly affect the blood flow at the target location. Therefore, the expandable member pressure B should be the same as the pressure from the proximal solid-state sensor 110a (T_{actual}).

B=T_{actual}:B∈B_{lowvol} (8)

Therefore, to calibrate the proximal solid-state sensor 110a, the controller 104 may determine a corrected proximal solid-state sensor offset as

∈′_{T}=T_{measured}−B (9)

where ∈′_{T} is the corrected proximal solid-state offset. The corrected proximal solid-state offset may be applied to the pressure measured at the proximal solid-state sensor 110a to determine a calibrated pressure value:

T_{calibrated}=T_{measured}−∈′_{T}=(T_{actual}+∈_{T})−∈′_{T} (10)

where T_{calibrated} may be the calibrated pressure value for the proximal solid-state sensor 111a, ∈_{T} may be the proximal solid-state offset in Equation 1, and ET may be the corrected proximal solid-state offset. Therefore, the calibration curve and/or calibration constant of the proximal solid-state sensor 110a may be adjusted by applying the correction factor ∈′_{T}. For instance, if the calibration curve of the proximal solid-state sensor 111a is represented as a straight line y=mx+c, where y may indicate the output voltage and x may indicate a change in pressure as measured and sensed by the distal solid-state sensor 111b, then the controller 104 may adjust and/or vary the calibration curve so that the output voltage y of the proximal solid state sensor 111a is the same as the output voltage of the expandable member sensor 115. For example, the calibration curve of the proximal solid-state sensor 111a may be adjusted to be y=mx+c+∈′_{T}.

When the volume of the calibration bolus is greater than the non-disruptive volume, the expandable member may begin to affect the blood flow at the target location. However, the expandable member pressure B′ may still be the same as the pressure from the proximal solid-state sensor 110a (T′_{actual}). Accordingly, when the volume of the calibration bolus is greater than the non-disruptive volume, the calibration curve and/or calibration constant of the proximal solid-state sensor 111a may still be adjusted in a manner similar to adjusting the calibration curve and/or calibration constant of the proximal solid-state sensor 111a when the volume of the calibration bolus equals the non-disruptive volume.

The controller 104 may adjust the calibration curve and/or the calibration constant of the proximal solid-state sensor 111a in the following ways. In some variations, the controller 104 may receive a pressure value from the expandable member sensor 115 at a specific time point. The controller 104 may adjust the calibration curve and/or the calibration constant of the proximal solid-state sensor 111a such that the pressure value from the proximal solid-state sensor 111a at that specific time point is the same as the pressure value from the expandable member sensor 115 at that time point. Additionally or alternatively, the controller 104 may receive sensor data in the form of a pressure waveform from the expandable member sensor 115. The controller may adjust the calibration curve and/or the calibration constant of the proximal solid-state sensor 111a such that the pressure waveform from the proximal solid-state sensor 111a is the same as the pressure waveform from the expandable member sensor 115. In some variations, the controller 104 may determine an average and/or mean of sensor data (e.g., pressure values) obtained from the expandable member sensor 115 for a given time duration. The controller 104 may then adjust the calibration curve and/or the calibration constant of the proximal solid-state sensor 111a such that the sensor data (e.g., pressure value) from the proximal solid-state sensor 111a is the same as the average and/or mean of the sensor data from the expandable member sensor 115.

In some variations, the distal solid-state sensor 111b and the proximal solid-state sensor 111a may be calibrated in a similar manner and as described above with respect to the proximal solid-state sensor 111a. In these variations, calibration of the proximal and distal solid-state sensors 111a, 111b may occur simultaneously or sequentially. In some variations, the distal solid-state sensor 111b may be calibrated based on the proximal solid-state sensor 111a after the proximal solid-state sensor 111a has been calibrated. For example, since the volume of the calibration bolus does not significantly affect the blood flow at the target location, the pressure from the proximal solid-state sensor (T_{actual}) should be the same as the pressure from the distal solid-state sensor 110a (H_{actual}). However, as discussed above, an inherent offset α may exist between the proximal solid-state sensor 111a and the distal solid-state sensor 111b. A corrected offset EH for the distal solid-state sensor 111b may therefore be determined as

∈′_{H}=∈′_{T}−α (11)

The corrected distal solid-state sensor offset (EH) may be applied to the pressure measured at the distal solid-state sensor 110b (H_{measured}) to determine a calibrated pressure value (H_{calibrated}) for the distal solid-state sensor 111b:

H_{calibrated}=H_{measured}−∈′_{H}=(H_{actual}+∈_{H})−∈′_{H} (12)

where H_{calibrated} may be the calibrated pressure value for the distal solid-state sensor 111b, H_{actual} may be the actual pressure value at the distal solid-state sensor 111b, EH may be the distal solid-state sensor offset, and EH may be the corrected distal solid-state sensor offset. Therefore, the calibration curve and/or calibration constant of the distal solid-state sensor 110b may be adjusted by applying the correction factor ∈′_{H}. Accordingly, the calibration curve of the distal solid-state sensor 111b may be represented as y=mx+c+∈′_{H}, where y may indicate the output voltage and x may indicate a change in pressure as measured and sensed by the distal solid-state sensor 111b, c may indicate the calibration constant for the distal solid-state sensor, and EH may indicate the constant that accounts for the pressure difference between the distal solid-state sensor 111b and the proximal solid-state sensor 111a. To calibrate the distal solid-state sensor 111b, the controller 104 may adjust and/or vary the calibration curve so that the output voltage y of the distal solid-state sensor 111b is the same as the output voltage of the proximal solid-state sensor 111a. Therefore, the controller 104 may adjust the calibration curve and/or the calibration constant of the distal solid-state sensor 111b to also account for the inherent pressure difference a between the proximal solid-state sensor 111a and the distal solid-state sensor 111b.

In some variations, the distal solid-state sensor 111b may be calibrated based on the proximal solid-state sensor 111a and an additional correction factor (e.g., post inflation offset β). For example, in some variations, as the expandable member 110 is injected with a volume greater than the non-disruptive volume, the sensor data from the proximal solid-state sensor 111a may be different from the sensor data from the distal solid-state sensor 111b. This is because when the volume is above the non-disruptive volume, the expandable member 110 may begin to affect the blood flow at the target location. For instance, when a calibration bolus that has a volume that is greater than the non-disruptive volume is injected into the expandable member 110, pressure proximal to or upstream of the expandable member 110 (e.g., pressure from the distal solid-state sensor 111b) may drop and/or may be lower than the pressure within the expandable member 110. However, pressure distal to or downstream of the expandable member 110 (e.g., pressure from proximal solid-state sensor 111a) may be same as the pressure within the expandable member 110. Since the expandable member 110 disrupts the pressure, the post inflation offset β discussed above as representing the offset between the proximal solid-state sensor 111a and the distal solid-state sensor 111b when the volume of the calibration bolus is above the non-disruptive volume may be used to calibrate the distal solid-state sensor 111b. Therefore, the calibration curve of the distal solid-state sensor 111b may be similar to the calibration curve of the proximal solid-state sensor 111b with the additional post inflation offset β representing the corrected difference in pressure measurement between the proximal solid-state sensor 111a and the distal solid-state sensor 111b.

Therefore, to calibrate the distal solid-state sensor 111b in this variation, the controller 104 may determine the corrected distal solid-state sensor offset as

∈″_{H}=∈″_{T}−β (13)

The corrected distal solid-state sensor offset (∈″_{H}) may be applied to the pressure measured at the distal solid-state sensor 110b (H′_{measured}) to determine a calibrated pressure value (H′_{calibrated}) for the distal solid-state sensor 111b:

H′_{calibrated}=H′_{measured}−∈″_{H}=(H′_{actual}+∈_{H})−∈″_{H} (14)

where H′_{calibrated} may be the calibrated pressure value for the distal solid-state sensor 111b, H′_{actual} may be the actual pressure value at the distal solid-state sensor 111b, ∈_{H} may be the distal solid-state sensor offset, and EH may be the corrected distal solid-state sensor offset. Therefore, the calibration curve and/or calibration constant of the distal solid-state sensor 110b may be adjusted by applying the correction factor EH. Accordingly, the calibration curve of the distal solid-state sensor 111b may be represented as y=mx+c+EH, where y may indicate the output voltage and x may indicate a change in pressure as measured and sensed by the distal solid-state sensor 111b, c may indicate the calibration constant for the distal solid-state sensor, and ∈″_{H} may indicate the constant that accounts for the pressure difference between the distal solid-state sensor 111b and the proximal solid-state sensor 111a. To calibrate the distal solid-state sensor 111b, the controller 104 may adjust and/or vary the calibration curve so that the output voltage y of the distal solid-state sensor 111b is the same as the output voltage of the proximal solid-state sensor 111a. Therefore, the controller 104 may adjust the calibration curve and/or the calibration constant of the distal solid-state sensor 111b to also account for the inherent pressure difference between the proximal solid-state sensor 111a and the distal solid-state sensor 111b.

In this manner, the controller 104 may be configured to calibrate the solid-state sensors 111 to accurately measure an absolute value (e.g., absolute pressure) from the proximal solid-state sensor 111a and the distal solid-state sensor 111b.

### Method of Calibrating Sensors

Generally, methods for calibrating solid-state sensors may include advancing an elongate body and an expandable member to a target location, injecting a calibration bolus into the expandable member, obtaining data (e.g., waveforms) from the expandable member sensor and one or more solid-state sensors, and adjusting the sensor data from at least one solid-state sensor based on data from the expandable member sensor. In some variations, vacuum may be applied to the expandable member (e.g., a balloon). The vacuum may be applied before or during the process (e.g., between any step) of calibrating the solid-state sensors, and may be used as a safeguard during calibration.

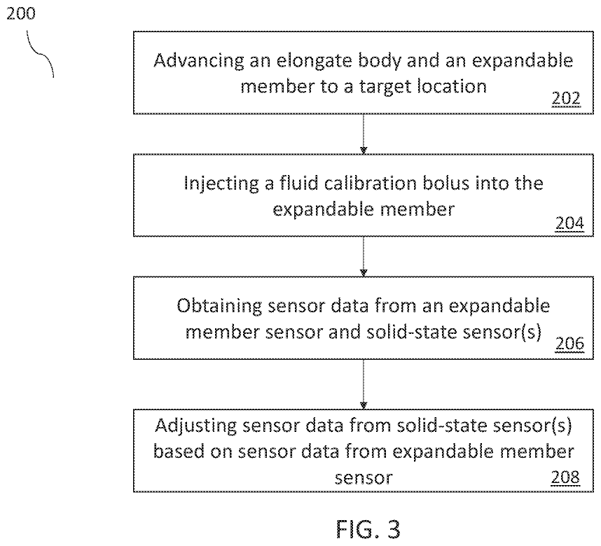

FIG. 3 is a flowchart illustrating an exemplary variation of a method 200 for calibrating solid-state sensors. The method 200 may include initially calibrating an expandable member sensor and/or at least one solid-state sensor prior to coupling the expandable member sensor and the at least one solid-state sensor to a system (e.g., system 100 in FIGS. 2A, 2B, and 2C). For example, the expandable member sensor and/or the at least one solid-state sensor may be factory calibrated. For example, initially calibrating the expandable member sensor and at least one solid-state sensor may include determining and/or setting a calibration curve and/or a calibration constant for each of the expandable member sensor and the at least one solid-state sensor as discussed herein.

Following initial calibration, the method 200 may include coupling the sensors to the system (e.g., system 100 in FIGS. 2A, 2B, and 2C) for measuring a physiological condition, which may also be used for calibrating one or more solid-state-sensors. For example, the expandable member sensor may be coupled to, contained in, attached to, or otherwise positioned in a controller, such as controller 104 in FIGS. 2A, 2B, and 2C. The at least one solid-state sensor may be coupled to (e.g., attached to, mounted on, affixed to, or otherwise integrated with) an elongate body (e.g., elongate body 102 in FIGS. 2A, 2B, and 2C). The elongate body may include an expandable member (e.g., expandable member 104 in FIGS. 2A, 2B, and 2C). The expandable member sensor may be fluidly coupled to the expandable member. For example, a fluid column within the elongate body may fluidly connect the expandable member sensor positioned in the controller to the expandable member. The at least one solid-state sensor may include two solid-state sensors, such as a distal solid-state sensor (e.g., distal solid-state sensor 111b in FIG. 2B) that may be positioned proximal to the expandable member and a proximal solid-state sensor (e.g., proximal solid-state sensor 111a in FIG. 2B) that may be positioned distal to the expandable member.

The method 200 may include, prior to using the system to monitor the patient or calibrate one or more solid-state sensors (e.g., prior to advancing a portion of the system into the patient's body), determining an initial setpoint for the expandable member sensor. For example, determining the initial setpoint may include determining a baseline value, such as a zero point for the expandable member sensor. For instance, the sensor reading measured from the expandable member sensor prior to advancing a portion the system may be set to zero (even if the reading itself does not show zero). Additionally or alternatively, determining the initial setpoint may include determining a zero point for the expandable member sensor based on the atmospheric pressure measured from a barometer.

The method 200 may include at 202 advancing a portion of the system (e.g., a portion of an elongate body and an expandable member) to a target location in a patient's body. In some variations, the elongate body or a portion thereof (e.g., tip or end portion) may be advanced to a target location in a blood vessel. For instance, the elongate body and the expandable member may be advanced to and inserted into an aorta via a suitable endovascular route. For example, the method may include inserting the end portion of the elongate body into the aorta through the femoral artery. In some variations, the elongate body may be inserted into the aorta through radial or brachial access. The elongate body may be advanced such that the expandable member is positioned at a desired location in the aorta. For example, the elongate body may be advanced until the expandable member is positioned in zone 1 of the aorta (e.g., the descending aorta above the celiac artery), zone 2 of the aorta (e.g., the abdominal aorta between the celiac artery and the lowest renal artery), or zone 3 of the aorta (e.g., the abdominal aorta between the lowest renal artery and the aortic bifurcation). Alternatively, the elongate body may be inserted into the iliac arteries and not advanced into the aorta.

When the portion of the elongate body and the expandable member are advanced to the target location in the patient's body, the at least one solid-state sensor (e.g., proximal solid-state sensor and distal solid-state sensor) may be exposed to fluids (e.g., bodily fluids such as blood) in the patient's body. More specifically, the at least one solid-state sensor may transition from a dry state (e.g., by exposure to air outside of the patient's body) to a wet state (e.g., by exposure to fluid inside the patient's body). This transition may result in a rapid change in sensor readings from the at least one solid-state sensor. For instance, the change in media, the change in temperature, and/or other changes in the surrounding conditions (surrounding the at least one solid-state sensor), may yield a rapid rise or a rapid drop in the sensor data. In some variations, the method may include determining that this transition has occurred and notifying a user (e.g., surgeon, operator, doctor, etc.) of this transition. For example, the method may include receiving sensor data from the at least one solid-state sensor and determining that the transition has occurred by identifying rapid changes in the sensor data as described in more detail herein. For example, methods may include analyzing pressure waveforms (e.g., arterial waveforms, venous waveforms, etc.) received from the at least one solid-state sensor, identifying a sudden change in gradient in the waveform, and determining that the transition has occurred. Methods may further include notifying a user, e.g., automatically upon the determination, of the transition (e.g., via a user interface and/or a display on the controller) based on this determination. Additionally or alternatively, the user may analyze sensor data being displayed on a user interface, such as a display on the controller. In response to observing a rapid rise or a rapid drop in the sensor data, the user may manipulate (e.g., press, push, pull, etc.) one or more controls (e.g., user interface with touchscreen buttons, physical buttons, etc.) on the controller to indicate to the controller that the transition has occurred. In some variations, determining that the transition has occurred may include determining that the expandable member has been positioned in the target blood vessel. In some variations, determining that the expandable member has been positioned in the target blood vessel may include analyzing sensor data such as determining Euclidian distance, performing Dynamic time-wrapping, performing root mean square similarity between sensor data from the proximal solid-state sensor and sensor data from the distal solid-state sensor. Identifying that the sensor data from the proximal solid-state sensor is substantially similar to the sensor data from the distal solid-state sensor (e.g., within +10 mmHg based on the analysis) may indicate that the expandable member has been positioned in the target blood vessel.

The method 200 may include, after advancing the expandable member to the target location, determining an inherent offset between the proximal solid-state sensor and the distal solid-state sensor. At 204, the method 200 may include injecting a calibration bolus into the expandable member. The calibration bolus may be injected via a pump (e.g., syringe pump 108 in FIGS. 2A and 2C). The pump may be fluidly coupled to the expandable member via a fluid column within the elongate body. In some variations, injecting the volume of the calibration bolus may include injecting a non-disruptive volume of fluid that does not distend the expandable member, but does fill the fluid column between the expandable member and the pump with the fluid. In such variations, the expandable member may not materially and/or substantially affect the blood flow (e.g., due to the difference in pressures measured by the proximal solid-state sensor and the distal solid-state sensor being not more than about 1.0 mmHg) at the target location. The pressure at the target location outside of the expandable member may be transduced via the fluid column to the expandable member sensor. In some variations, injecting the calibration bolus may include injecting a fluid volume that is greater than the non-disruptive volume, but lower than the upper volume limit. Thus, the calibration bolus may begin to disrupt the blood flow at the target location. However, the calibration bolus may not result in pressure exertion at the target location such that the pressure inside the expandable member may still be the same as or may be substantially similar to (e.g., within ±10 mmHg) the pressure outside the expandable member at the target location. In some variations, when the volume of the calibration bolus is greater than the non-disruptive volume (but below the upper volume limit), the method may further include determining a post inflation offset β between the proximal solid-state sensor and the distal solid-state sensor as discussed in detail above. In some variations, the volume of the calibration bolus, and in some variations, the non-disruptive volume, may be between about 1 ml to about 1.5 ml.

Prior to injecting the calibration bolus, the method 200 may also include determining the non-disruptive volume. The non-disruptive volume may be determined based on one or more of: size of the expandable member 110, shape of the expandable member 110, volume of the expandable member 110, length of the elongate body 102, volume of the fluid column that fluidly couples and/or connects the expandable member 110 with the pump 108, length of fluid column coupling the expandable member 110 with the pump 108, amount of fluid needed to transition negative pressure readings obtained from the expandable member sensor 115 to zero pressure reading and/or a positive pressure reading, and size of the target blood vessel including a combination thereof, as described in more detail above.

The method 200 may also include determining a time point at which the calibration bolus is to be injected into the expandable member. The time point may be determined based on one or more of: length of the elongate member, distance of the solid-state sensors 111 from the distal end of the elongate member, length of the fluid column that fluidly couples and/or connects the expandable member with the pump 108, sensor data (e.g., waveforms) from the proximal solid-state sensor and distal solid-state sensor, including one or more thereof.

The method 200 may also include determining whether the volume of the calibration bolus equals the non-disruptive volume or a volume greater than the non-disruptive volume. For instance, the method 200 may include determining whether the non-disruptive volume has been injected into the expandable member and/or verifying whether the non-disruptive volume has been injected into the expandable member. For example, verifying that the non-disruptive volume of fluid has been injected may include analyzing the sensor data from the expandable member sensor to determine whether the sensor measurement has transitioned from a negative value to a zero value and/or a positive value. If the sensor measurement transitions to a zero value and/or a positive value, the method may include optionally notifying the user that the non-disruptive volume of fluid has been injected.

In some variations, verifying that the non-disruptive volume of fluid has been injected into the expandable member may include comparing sensor data from each of the solid-state sensors with the remainder of the solid-state sensors. For example, if the elongate body includes a proximal solid-state sensor and a distal solid-state sensor, the method may include comparing the sensor data from the proximal solid-state sensor to the sensor data from the distal solid-state sensor. For instance, in response to determining that a change in gradient in the pressure waveform from the proximal solid-state sensor is the same as a change in gradient in the pressure waveform from the distal solid-state sensor, the method may include determining that the desired volume of fluid has been injected into the expandable member. The method may optionally include notifying the user that the non-disruptive volume of fluid has been added.

In some variations, verifying that the non-disruptive volume of fluid has been added into the expandable member may include comparing the sensor data from the expandable member sensor with the sensor data from the at least one solid-state sensor. For example, in response to determining that a change in gradient in the pressure waveform from the expandable member sensor is the same as or substantially similar to (e.g., within +10 mmHg) a change in gradient in the pressure waveform from both the proximal solid-state sensor and the distal solid-state sensor, the method may include determining that the non-disruptive volume of fluid has been injected into the expandable member. The method may optionally include notifying the user that the non-disruptive volume of fluid has been added.

Additionally or alternatively, the method may include determining that a volume greater than the non-disruptive volume of fluid has been injected into the expandable member. For instance, if a change in gradient in the pressure waveform received from one of the solid-state sensors is different from a change in gradient in the pressure waveform received from the remainder of the solid-state sensors, the method may include determining that a volume above the non-disruptive volume has been added to the expandable member. The method may optionally include notifying the user that the volume above the non-disruptive volume has been added. As another example, if a change in gradient in the pressure waveform received from the expandable member sensor is the same as a change in gradient in the pressure waveform received from the proximal solid-state sensor, and different from a change in gradient in the pressure waveform received from the distal solid-state sensor, the method may include determining that volume above the non-disruptive volume has been added to the expandable member. The method may optionally include notifying the user that the volume above the non-disruptive volume has been added.

After determining that the calibration bolus has been injected into the expandable member, the method may include calibrating the at least one solid-state sensor. In some variations, the calibration may begin after a predetermined amount of time has elapsed after the elongate member has been advanced into the target location in the patient's body. The predetermined time may be preprogrammed into the controller and/or may be received by the controller from a user, e.g., using the user interface. In some variations, the method may include notifying the user indicating that the predetermined time has elapsed and/or that the controller may automatically begin to calibrate the at least one solid-state sensor. Additionally or alternatively, the user may manipulate one or more controls on the controller to indicate that the predetermined time has elapsed, the calibration bolus has been added, and/or that the controller may begin to calibrate the at least one solid-state sensor. For example, the user may manipulate a button (e.g., user interface on a touch screen and/or physical button) to instruct the controller to start the calibration of the at least one solid-state sensor.

At 208, the method 200 may include adjusting sensor data from the at least one solid-state sensor based on the sensor data from the expandable member sensor. For example, the method may include shifting and/or adjusting a calibration curve and/or a calibration constant of the proximal solid-state sensor such that the sensor data from the proximal solid-state sensor is the same as the sensor data from the expandable member sensor. In some variations, the calibration curve of the proximal solid-state sensor may be adjusted such that the sensor data at a specific data point or points from the proximal solid-state sensor is the same as the sensor data from the expandable member sensor at the same data point or points. In some variations, adjusting the calibration curve of the proximal solid-state sensor may include determining a mean of the sensor data from the proximal solid-state sensor and a mean of the sensor data from the expandable member sensor. The calibration curve of the proximal solid-state sensor may be adjusted such that the mean of the sensor data from the proximal solid-state sensor is the same as the mean of the sensor data from the expandable member sensor. In some variations, the calibration curve of the proximal solid-state sensor may be adjusted such that the pressure waveform from the proximal solid-state sensor is the same the pressure waveform from the expandable member sensor.

In some variations, calibrating the distal solid-state sensor may include calibrating the distal solid-state sensor in a similar manner as described above with respect to the proximal solid-state sensor. In these variations, the proximal and the distal solid-state sensors may be calibrated simultaneously or sequentially. In some variations, the method may include adjusting and/or shifting the calibration curve and/or the calibration constant of the distal solid-state sensor based on the calibrated proximal solid-state sensor and the inherent offset α between the proximal solid-state sensor and the distal solid-state sensor.

In some variations, the method may include adjusting and/or shifting the calibration curve and/or the calibration constant of the distal solid-state sensor based on the calibrated proximal solid-state sensor and the post inflation offset β between the proximal solid-state sensor and the distal solid-state sensor.

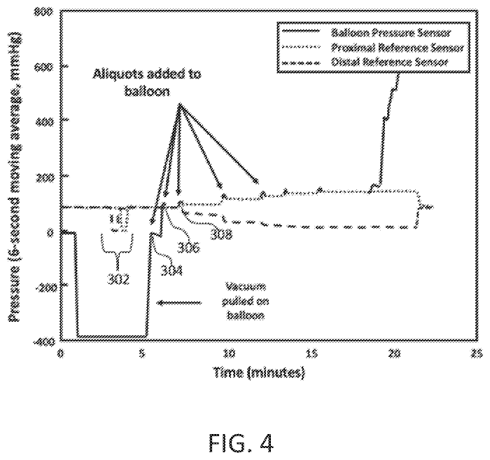

FIG. 4 illustrates an exemplary variation of sensor data from an expandable member sensor, a proximal solid-state sensor, and a distal solid-state sensor before calibration, during calibration, and after calibration using the systems and methods described herein. In this exemplary variation, the volume of the calibration bolus is the non-disruptive volume. At 302 (at about 3 minutes) the solid-state sensors may experience a transition from air to water. The drop in the pressure values from the solid-state sensors indicate this transition. At 304 (at about 5 minutes), an aliquot calibration bolus may be added. This aliquot may be enough to overcome the partial vacuum in the expandable member sensor. Accordingly, the pressure value from the expandable member sensor moves from a negative value to zero. At 306, a second aliquot of fluid may be added. The total of the fluid added at 304 and fluid added at 306 may be the calibration bolus. Accordingly, at 306 the solid-state sensors are calibrated based on the sensor data from the expandable member sensor. At 306, the sensor values from both the solid-state sensors match the sensor value from the expandable member sensor. At 308, excess fluid is added. At this point, the pressure value from the proximal solid-state sensor may fall away from the pressure value from the distal solid-state sensor.

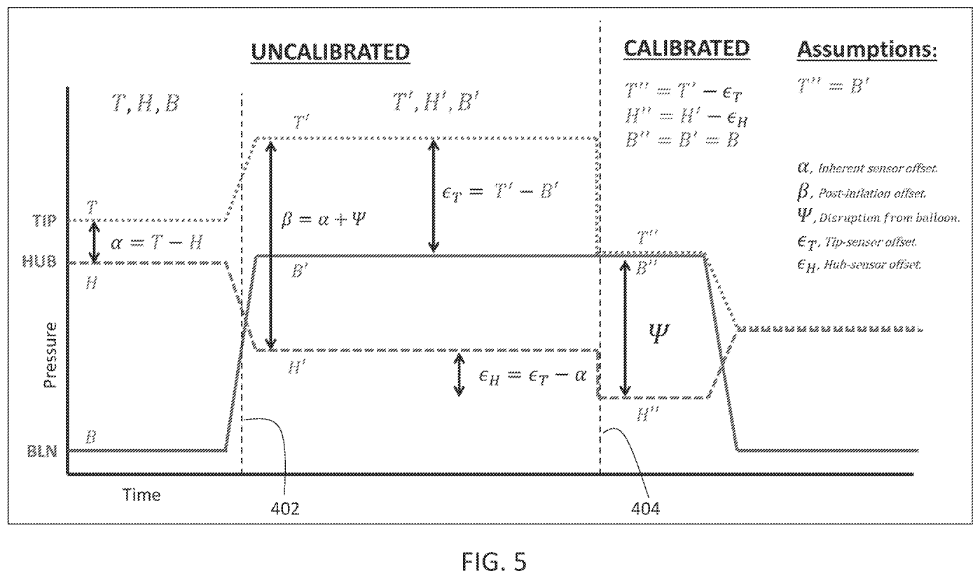

FIG. 5 illustrates another exemplary variation of sensor data from an expandable member sensor, a proximal solid-state sensor, and a distal solid-state sensor before calibration, during calibration, and after calibration using the systems and methods described herein. In this variation, the volume of the calibration bolus is above the non-disruptive volume. The sensor data shown is data collected after the solid-state sensors experience the transition of media. After the transition of media and application of negative pressure to the expandable member (measured by expandable member sensor BLN), but before the addition of the calibration bolus (e.g., at 402), the inherent offset α between the proximal solid-state sensor (i.e., tip sensor) and the distal solid-state sensor (i.e., hub sensor) may be determined. As indicated in FIG. 5, before 402, an inherent offset α may exist between pressure values measured from the proximal solid-state sensor (T) and the distal solid-state sensor (H). The expandable member pressure measured by the expandable member sensor (BLN) may be negative due to vacuum applied to the expandable member as described in more detail herein. At 402, the calibration bolus, which as noted above, has a volume greater than the non-disruptive volume of fluid, may be added to the expandable member. Therefore, at 402, the expandable member pressure (B) may rise to a positive value (B′). A change in gradient in the waveform from the proximal solid-state sensor may be similar to the change in gradient in the waveform from the expandable member sensor. However, since the volume of the calibration bolus is greater than the non-disruptive volume, the pressure value from the distal solid-state sensor may begin to fall. That is, the change in gradient in the waveform from the distal solid-state sensor may be different from the change in gradient in the waveform from the expandable member sensor. Therefore, in some variations, after 402, a post inflation offset β between the proximal solid-state sensor and the distal solid-state sensor may be determined. In these variations, the post inflation offset β may be determined based on the inherent offset α and the disrupted pressure Ψ due to the expandable member.

The sensor data from the expandable member sensor may be used to calibrate the proximal solid-state sensor. A corrected proximal solid-state sensor offset (e.g., corrected proximal solid-state sensor offset ∈′_{T} described above) may be calculated so that the sensor data from the proximal solid-state sensor matches the sensor data from the expandable member sensor. This corrected proximal solid-state sensor offset may be applied at 404. As seen at 404, the sensor data from the proximal solid-state sensor begins to match the sensor data from the expandable member sensor due to the application of the corrected proximal solid-state sensor offset. However, since the sensor data from the distal solid-state sensor begins to fall away at 402 (due to the expandable member), the corrected distal solid-state sensor offset (e.g., corrected distal solid-state sensor offset ∈″_{H} and/or corrected distal solid-state sensor offset ∈′_{H} described above) may be calculated such that the inherent offset α between the proximal solid-state sensor and the distal solid-state sensor may be considered. This corrected distal solid-state sensor offset may be applied at 404.

In other variations, as previously mentioned, the post inflation offset β may not be calculated as part of the calibration process. In these variations, the equations are the same as previously discussed herein (using the same equation numbers used above), and the distal solid-state sensor offset may be determined directly based on the inherent offset and the proximal solid-state sensor offset, as described above. Accordingly, the pressure measured at the proximal solid-state sensor and distal solid-state sensor (i.e., the tip sensor and the hub sensor, respectively) may be the actual pressure at the respective sensors plus an unknown amount of constant error (ϵ (T, H)), exemplified as:

T_{measured}=T_{actual}+∈_{T} (1)

H_{measured}=H_{actual}+∈_{H} (2)

The pressure (B) inside of the expandable member (e.g., balloon (BLN)) may be considered as the blood pressure at the proximal solid-state sensor (tip sensor (T_{actual})) when the expandable member is inflated with a volume of fluid that may translate the upstream blood pressure through the expandable member membrane (γ_{lower}) but not affect the overall pressure inside the expandable member (γᵤₚₚₑᵣ). This set of expandable member pressures may be referred to as B_{lowvol}:

∀B: B=balloon pressure (5)

B_{lowvol}={B:γ_{lower}<B and B<γᵤₚₚₑᵣ and γ_{lower}>0} (6)

B=T_{actual}:B∈B_{lowvol} (8)

When vacuum has been applied to the expandable member, it usually does not interfere with blood flow and thus, the blood pressure at the proximal solid-state sensor (tip sensor) and distal solid-state sensor (hub sensor) are generally the same:

When B<0, T_{actual}=H_{actual} (3)

Equations (1) and (2) may then be used to estimate the inherent sensor offset relative to each other (α):

α=T_{measured}−H_{measured}=∈_{T}−∈_{H} (4)

When the balloon pressures are within B_{lowvol}, from Equation (8) it is known that the balloon pressure may match the actual blood pressure at the tip. Therefore, Equations (1) and (8) may be used to obtain an estimate of the tip sensor error (ϵ′_{T}≈ϵ_{T}) that may be corrected using pressure measurements at the proximal solid-state sensor (tip sensor) and expandable member sensor (BLN):

∈′_{T}=T_{measured}−B (9)

Estimating the distal solid-state sensor (hub sensor) error (ϵ′_{H}≈ϵ_{H}) may then be accomplished by combining Equations (4) and (9):

∈′_{H}=∈′_{T}−α (11)

Next, the software of the control system may apply a correction factor to both T_{measured} and H_{measured} using the estimates of the tip and hub sensor errors to calibrate the sensors:

T_{calibrated}=T_{measured}−∈′_{T}=(T_{actual}+∈_{T})−∈′_{T} (10)

H_{calibrated}=H_{measured}−∈′_{H}=(H_{actual}+∈_{H})−∈′_{H} (12)

Vacuum (negative pressure) may be applied to the expandable member (e.g., a balloon) before or during any step of the process of calibrating the solid-state sensors, and may be used as a safety feature during calibration, as previously mentioned above. In some variations, the vacuum may be applied to avoid introducing an air bubble into the elongate member shaft. Air bubbles may be incidentally introduced while coupling and/or decoupling syringes (e.g., at the stopcock that couples the syringe to the expandable member, such as from dead space within the stopcock). Air bubbles may have an adverse impact on the pressures being transduced from the expandable member through the elongate member to the expandable member sensor. For example, in an elongate member filled with an incompressible fluid (e.g., water, saline), the pressure applied at the expandable member at one end of the elongate member generally transduces fully to the expandable member sensor at the opposite end of the elongate member. However, when an air bubble exists between the expandable member and the expandable member sensor, the air in that bubble may compress and ineffectively transduce the pressures from the expandable member to the expandable member sensor. Thus, by applying negative pressure to the expandable member (e.g., at the start of the calibration process) as a first calibration safeguard, the chance of having air bubbles in the elongate member when a calibration bolus is applied may be minimized, which in turn may reduce the chance of error in calibration of the solid-state sensors. In some variations, the amount of negative pressure that may be applied to the expandable member may range from about −50 mmHg to about −350 mmHg, including all values and sub-ranges therein. For example, the applied negative pressure may be about −50 mmHg, about −75 mmHg, about −100 mmHg, about −125 mmHg, about −150 mmHg, about −175 mmHg, about −200 mmHg, about −225 mmHg, about −250 mmHg, about −275 mmHg, about −300 mmHg, about −325 mmHg, or about −350 mmHg. In some variations, the applied negative pressure may be about −275 mmHg.

Negative pressure may be applied to the expandable member using a syringe pump. The syringe pump may be operated manually (e.g., actuated by a user by hand, without use of the controller), or by using the controller. The syringe pump may be the same pump used to deliver the calibration bolus and/or the same pump used to modify the volume of the expandable member (e.g., inflate and/or deflate) to control blood flow. Alternatively, the syringe pump used to apply the negative pressure may be a different syringe pump than the pump used to deliver the calibration bolus and/or modify the volume of the expandable member (e.g., three different syringe pumps may be used, one for applying negative pressure, one for delivering the calibration bolus, and one for modifying the volume of the expandable member). The syringe pump used to apply the negative pressure may be decoupled from the system and replaced with a different syringe pump that may be used to inject the calibration bolus and/or modify the volume of the expandable member. In variations where the syringe pump is used to apply negative pressure, the syringe pump may have a volume between about 1.0 ml to about 10 ml, including all values and sub-ranges therein. For example, the syringe pump may have a volume of about 1.0 ml, about 2.0 ml, about 3.0 ml, about 4.0 ml, about 5.0 ml, about 6.0 ml, about 7.0 ml, about 8.0 ml, about 9.0 ml, or about 10 ml. In one variation, the syringe pump has a volume of about 3.0 ml.

Additional calibration safeguards (e.g., a second, third, fourth calibration safeguard) may be included during the calibration process of the solid-state sensors to prevent a user (e.g., surgeon, operator, etc.) from incorrectly proceeding through the calibration steps. These calibration safeguards may be used to check the accuracy of expandable member (e.g., balloon) pressure values at different stages of the calibration process, and may prevent movement to a subsequent stage when predetermined pressure values are not met. In this instance, the user (e.g., surgeon, operator, etc.) may be prompted by the system to perform a required action. The additional calibration safeguards may include the application of vacuum to the expandable member prior to measuring the inherent sensor offset to check that the expandable member volume is zero (e.g., by checking that pressure in the expandable member is less than about 5-10 mmHg), and that the values at the proximal and distal solid-state sensors are not impacted by any expandable member fluid. Other calibration safeguards may be included in the process prior to measuring the tip sensor offset. For example, the system may include a calibration safeguard that checks whether the expandable member pressure is at or above a threshold value, e.g., a non-negative value (zero or positive value) before measuring the tip sensor offset. This may assist in ensuring that enough fluid is in the expandable member/elongate member to transduce physiological pressures to the expandable member sensor.

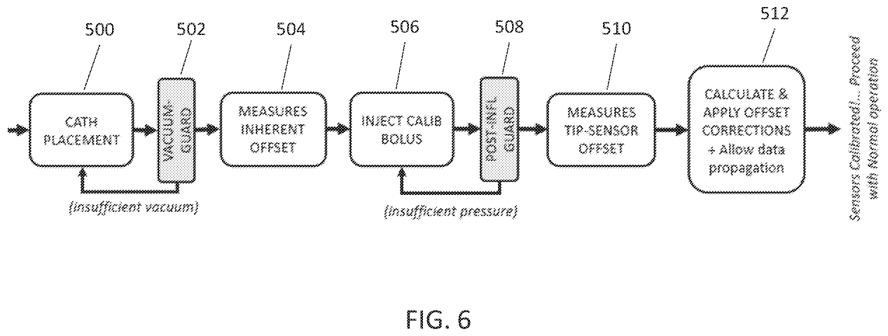

Referring to FIG. 6, an exemplary calibration process is illustrated after placement of an elongate member including an expandable member in a bodily lumen (e.g., blood vessel). After the elongate member (e.g., a catheter) is placed in step 500, vacuum may be applied as a calibration safeguard in step 502 (vacuum guard) to ensure the expandable member pressure is at or below zero. If there is insufficient vacuum, the user may be prompted by the system to repeat vacuum application 502 until the expandable pressure measures zero or below zero. The inherent offset between the solid-state sensors (e.g., proximal and distal solid-state sensors) may then be measured in step 504, and a calibration bolus may be injected in step 506. Prior to measuring the tip sensor offset in step 510, another calibration safeguard 508, a post-inflation guard (post-infl guard), may be included to confirm that expandable member pressure is non-negative (e.g., zero or positive). The post-inflation guard may serve as a check to ensure that enough fluid is in the expandable member/elongate member to transduce physiological pressures to the expandable member sensor. If the expandable member pressure is found to be insufficient (e.g., negative), the user may be prompted by the system to repeat the injection of the calibration bolus 506 until the expandable member pressure is non-negative. After all calibration safeguards have been met, the system may then proceed in step 512 to calibrate the solid-state sensors using the calculated inherent and tip sensor offsets.

Although the elongate body is described herein as including an expandable member, it should be readily understood that the elongate body need not comprise an expandable member. In variations in which an expandable member is not used, a sensor of the first sensor type, which provides sensor data that may be used to calibrate the solid-state sensors, may be a fluid-column based pressure sensor that is integrated into the elongate body without an expandable member. For example, the fluid column may terminate at a point along the elongate body, such as, for example, near the distal end of the elongate body. The elongate body may include a non-expandable window (e.g., hole) fluidly coupled to the fluid column. The window may comprise a thin film such that the pressure surrounding and/or outside the non-expandable window may be transduced via the fluid column to the fluid-based pressure sensor without the use of an expandable member. To calibrate the solid-state sensors, the fluid column may be filled with the calibration bolus. The pressure around the elongate body may be transduced via the fluid column and the sensor readings from the fluid-column based pressure sensor may be used to the adjust the sensor readings from the solid-state pressure sensors, thereby calibrating the solid-state sensors based on sensor data from the fluid-column based sensor.

While the first sensor may be any type of sensor suitable for use in calibrating the solid-state sensors, utilizing the expandable member sensor as described herein to calibrate the solid-state sensors has many advantages, especially in variations in which an expandable member is necessary for blood flow control during patient treatment. For instance, utilizing the expandable member sensor, which for blood flow control devices, may already be incorporated into the device for use during patient treatment, to calibrate the solid-state sensors allows for the size of the device to remain unchanged and simplifies device construction and use, since additional components (e.g., sensors, fluid-columns, etc.) are not needed. Put another way, utilizing the expandable member sensor to calibrate the solid-state sensors eliminates the need for additional components solely used for calibration. Instead, the expandable member sensor may be used both to assist in controlling blood flow and for calibrating the solid-state sensors, thus removing the need for calibration-specific components that may enlarge the device and/or make it more difficult to make and/or use.

After the solid-state sensors have been calibrated, the devices, systems and methods described herein may be used to control blood flow in various locations in the body using the sensor data from the calibrated solid-state sensors. Each of the calibrated solid-state sensors may measure patient physiologic information, such as physiologic information indicative of blood pressure or blood flow through a blood vessel (e.g., the aorta), to determine the patient's underlying physiology. In some variations, the sensor data from the calibrated sensors is used to control blood flow in the patient. For example, the calibrated sensor data may be used to determine the size of the expandable member (e.g., determine an amount of fluid and/or compressed gas to be injected into or removed from the expandable member) so as to adjust the size of the expandable member and thereby affect blood flow.

While described above in relation to use in the aorta, it should be readily understood that the devices, systems, and methods described herein may be used in various vascular procedures such as interventions on arteries such as coronary arteries or cerebral arteries (e.g., thrombectomy procedures for stroke patients, etc.). In some variations, the devices, systems, and methods may be used to control blood flow in order to treat a patient suffering from shock such as neurogenic, hemorrhagic, hypovolemic, and/or septic shock. For example, the devices, systems, and methods may utilize the expandable member to partially occlude the blood vessel of a patient, which may provide blood pressure support to vascular beds above the expandable member while permitting continued perfusion distal to the expandable member. Thus, the devices, systems, and methods described herein may result in a more physiologic augmentation of proximal blood pressure, while also reducing ischemic injury to distal tissues by permitting continued perfusion distal to the expandable member.

### EXAMPLES

The following example is illustrative only and should not be construed as limiting the disclosure in any way.

### Example 1: Calibration of Solid-State Sensors

Data was captured during an animal experiment (pig) using a catheter that incorporated a balloon sensor and solid-state pressure sensors both proximal and distal to the balloon. External reference sensors were also placed inside of the animal that match the locations of the proximal and distal solid-state sensors on the catheter (when the catheter is inserted into the animal), and their data recorded using an external data acquisition system (PowerLab). The data measured by the catheter was synchronized in real-time with the external sensor data.

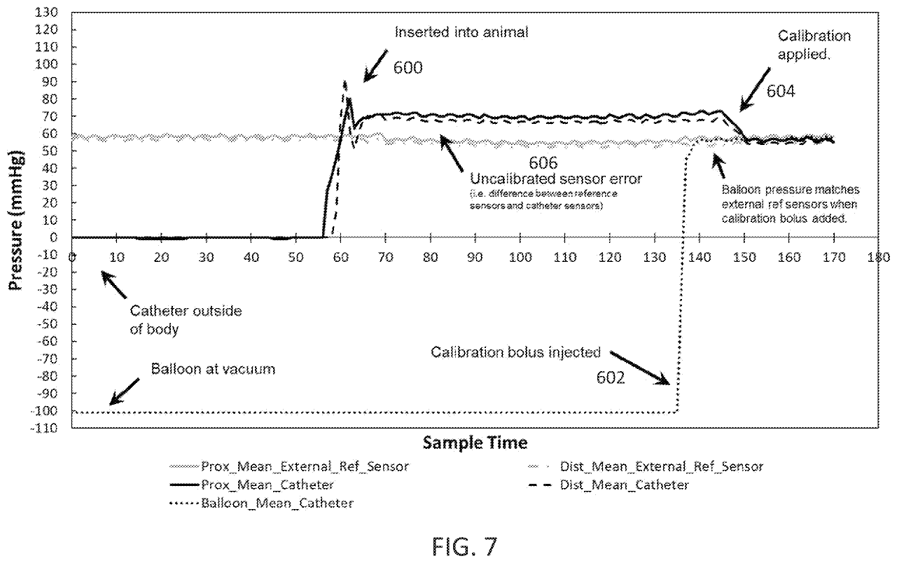

In this experiment, the volume of the calibration bolus was a non-disruptive volume of 2.0 ml. Referring to FIG. 7, at 600 (at about 60 minutes) the solid-state sensors were inserted into the pig aorta, transitioning from air to blood. The increase in the pressure values from the solid-state sensors (Prox_Mean_Catheter and Dist_Mean_Cath) indicated this transition. Pressure from the external reference sensors (Prox_Mean External_Ref_Sensor and Dist_Mean_External_Ref_Sensor) was measured to be about 60 mmHg. At 602 (at about 135 minutes), a calibration bolus was added. The volume of the calibration bolus was enough to overcome the partial vacuum in the balloon. Accordingly, the pressure value from the balloon sensor (Balloon_Mean_Cath) moved from a negative value (−100 mmHg) to about 60 mmHg, which matched the external reference sensors. Thereafter, at 604 the solid-state sensors were calibrated to remove the uncalibrated sensor error 606 based on the sensor data from the balloon sensor so that the sensor values from both solid-state sensors matched the sensor value from the balloon sensor.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method for calibrating at least one solid-state sensor coupled to an elongate body comprising an expandable member, the method comprising:
advancing the expandable member to a target location in a blood vessel of a patient;
injecting a fluid calibration bolus into the expandable member;
obtaining, using a controller, a first sensor data from an expandable member sensor, the first sensor data representing pressure in the expandable member;
obtaining, using the controller, a second sensor data from the at least one solid-state sensor; and
adjusting the second sensor data based on the first sensor data.

2. The method of claim 1, wherein adjusting the second sensor data comprises shifting at least one of a calibration curve and a calibration constant of the at least one solid-state sensor.

3. The method of claim 1, wherein adjusting the second sensor data further comprises:
determining a first mean of the first sensor data;
determining a second mean of the second sensor data; and
adjusting the second mean based on the first mean.

4. The method of claim 1, wherein the first sensor data includes a first waveform and the second sensor data includes a second waveform, and wherein adjusting the second sensor data comprises adjusting the second waveform based on the first waveform.

5. The method of claim 1, wherein the second sensor data and the first sensor data comprises data from the at least one solid-state sensor and data from the expandable member sensor respectively at a point in time.

6. The method of claim 1, wherein the pressure in the expandable member is indicative of pressure at the target location in the blood vessel.

7.-10. (canceled)

11. The method of claim 1, wherein the volume of the fluid calibration bolus is between about 1 ml and about 1.5 ml.

12. The method of claim 1, wherein the volume of the fluid calibration bolus is less than 5% of a volume of the expandable member.

13. The method of claim 1, wherein the volume of the fluid calibration bolus does not distend the expandable member.

14.-22. (canceled)

23. The method of claim 1, wherein the second sensor data from the at least one solid-state sensor includes a first waveform, the method further comprising determining, based at least in part on the first waveform, whether a volume of the fluid calibration bolus is a non-disruptive volume of fluid.

24.-25. (canceled)

26. The method of claim 1, wherein the at least one solid-state sensor includes a first pressure sensor proximal to the expandable member and a second pressure sensor distal to the expandable member.

27. The method of claim 26, wherein adjusting the second sensor data includes:
adjusting sensor data from the second pressure sensor based on the first sensor data; and
adjusting sensor data from the first pressure sensor based on the first sensor data.

28. The method of claim 26, wherein the second sensor data includes third sensor data from the first pressure sensor and fourth sensor data from the second pressure sensor, and wherein adjusting the second sensor data includes:
adjusting fourth sensor data from the second pressure sensor based on the first sensor data; and
adjusting third sensor data from the first pressure sensor based on the fourth sensor data.

29. The method of claim 1, further comprising one or more calibration safeguards.

30. The method of claim 29, wherein the one or more calibration safeguards comprises applying negative pressure to the expandable member prior to injecting the fluid calibration bolus.

31. The method of claim 30, wherein the negative pressure is applied using a syringe pump.

32. The method of claim 31, wherein the syringe pump is also used to inject the fluid calibration bolus into the expandable member.

33.-34. (canceled)

35. The method of claim 29, wherein the one or more calibration safeguards comprises measuring the pressure in the expandable member after injecting the fluid calibration bolus.

36. The method of claim 35, further comprising repeating injection of the fluid calibration bolus if the measured pressure in the expandable member is negative.

37. The method of claim 1, wherein the elongate body and the expandable member are used to control blood flow in a patient.

38. The method of claim 37, wherein controlling blood flow is used to treat one or more types of shock selected from the group consisting of neurogenic shock, hemorrhagic shock, hypovolemic shock, and septic shock.

39. (canceled)

40. A system for measuring a physiological condition in a patient comprising:
an elongate body comprising an expandable member and a solid-state sensor;
a syringe pump in fluid communication with the expandable member; and
a controller comprising an expandable member sensor in fluid communication with the expandable member, wherein the controller is communicatively coupled to the at least one solid-state sensor and configured to:
obtain, from the expandable member sensor, a first sensor data representing pressure at a target location in the patient;
obtain, from the solid-state sensor, a second sensor data; and
adjust the second sensor data based at least in part on the first sensor data.

41.-60. (canceled)

61. The system of claim 40, wherein the controller is further configured to implement one or more calibration safeguards.

62. A method for calibrating a first solid-state sensor and a second solid-state sensor coupled to an elongate body comprising an expandable member, the method comprising:
obtaining, using a controller, a first sensor data from the first solid-state sensor, wherein the first solid-state sensor is positioned distal to the expandable member;
obtaining, using the controller, a second sensor data from the second solid-state sensor, wherein the second solid-state sensor is positioned proximal to the expandable member;
determining, using the controller, an inherent offset between the first solid-state sensor and the second solid-state sensor;
injecting a calibration bolus into the expandable member;
obtaining, using the controller, a third sensor data from an expandable member sensor, the third sensor data representing pressure at the target location;
adjusting the first sensor data based on the third sensor data; and
after adjusting the first sensor data, adjusting the second sensor data based on the first sensor data and the inherent offset.

63.-70. (canceled)
